(19) United States
(12) Patent Application Publication
(10) Pub. No.: US 2014/0236152 A1
(43) Pub. Date: Aug. 21, 2014

(54) ELECTROSURGICAL DEVICE AND METHODS OF MANUFACTURE AND USE

(71) Applicants: Erik Walberg, San Jose, CA (US); Brandon Loudermilk, San Francisco, CA (US); Timothy Koss, Palo Alto, CA (US); Douglas C. Limbach, Palo Alto, CA (US); Mark Stirrat, Palo Alto, CA (US)

(72) Inventors: Erik Walberg, San Jose, CA (US); Brandon Loudermilk, San Francisco, CA (US); Timothy Koss, Palo Alto, CA (US); Douglas C. Limbach, Palo Alto, CA (US); Mark Stirrat, Palo Alto, CA (US)

(73) Assignee: Aesculap AG, Tuttlingen (DE)

(21) Appl. No.: 14/239,634

(22) PCT Filed: Aug. 22, 2012
(86) PCT No.: PCT/EP2012/066356

§ 371 Date: Apr. 22, 2014

Related U.S. Application Data
(60) Provisional application No. 61/526,679, filed on Aug. 23, 2011.

(51) Int. Cl. A61B 18/14

(52) U.S. Cl. CPC A61B 18/1445
USPC 606/52

(57) ABSTRACT

Embodiments of the disclosed technology relate to bipolar electrosurgical devices, as well as methods of manufacture and use of such devices. Embodiments of the device may include a set of opposing jaws comprising at least one bipolar electrode pair disposed thereon, the set of jaws configured to deliver radiofrequency energy to a target tissue. In some embodiments, a standoff member is provided to maintain a physical gap between the pair(s) of electrodes.

All publications and patent applications mentioned in this specification are herein incorporated by reference to the same extent as if each such individual publication or patent application were specifically and individually indicated to be so incorporated by reference.

### TECHNICAL FIELD

The disclosed technology relates to devices, systems and methods for electrosurgery. More particularly, the technology relates to jaw structures for such devices.

### BACKGROUND

Biopolar electrosurgical instruments apply radiofrequency (RF) energy to a surgical site to cut, ablate, or coagulate tissue. A particular application of these electrosurgical effects is to seal blood vessels or tissue sheets. A typical instrument takes the form of a set of forceps or pair of jaws, with one or more electrodes on each jaw tip. In an electrosurgical procedure, the electrodes are placed in close proximity to each other as the jaws are closed on a target site such that the path of alternating current between the two electrodes passes through tissue within the target site. The mechanical force exerted by the jaws and the electrical current combine to create the desired surgical effect. By controlling the level of mechanical and electrical parameters, such as the pressure applied by the jaws, the gap distance between electrodes, and the voltage, current, frequency, and duration of the electrosurgical energy applied to the tissue, the surgeon can coagulate, cauterize, or seal tissue toward a therapeutic end.

Electrosurgical procedures can be performed in an open environment, through conventional incisions, or they may be performed laparoscopically, through small incisions, typically 0.5 cm-1.5 cm in length. A laparoscopic procedure may include the use of a telescopic rod lens system that is connected to a video camera and to a fiber optic cable system that conveys light to illuminate the operative field. A laparoscope is typically inserted into a port in the body through a 5 mm or 10 mm cannula or trocar to view the operative field. Surgery is performed during a laparoscopic procedure with any of various tools that are typically arranged at the distal end of a shaft and are operable by manipulation of a handle or an actuator positioned at the proximal end of the shaft, and are dimensioned such that they can pass through a port provided by the 5 mm or 10 mm cannula.

As electrosurgical tools are applied in laparoscopic procedures, challenges to the devices arise regarding dimensional constraints imposed by the operating environment, including the smallness of a typical port of entry, which includes the use of conventional trocars with a 5 mm inner diameter. The technology provided herein addresses the need for improvements in device technology, that permit downsizing of the device while maintaining appropriate levels of mechanical strength and electrosurgical capability. For example, it is generally desirable to extend the length of conventional forceps in order to allow the sealing of greater lengths of tissue. As forceps length increases, it becomes a challenge to exert an appropriate level of force, particularly from the distal end of the forceps. The present disclosure provides technologies that represent progress in addressing challenges with electrosurgical devices, systems and methods.

### SUMMARY OF THE DISCLOSURE

Embodiments of the technology relate to an electrosurgical device that is particularly suitable for laparoscopic procedures in that its distal insertable portion, including a shaft and an end effector, may have a diameter no wider than about 5 mm. This 5 mm insertable profile allows insertion of the device through a conventional 5 mm trocar. Commercially available trocars that are conventionally referred to as being “5 mm” generally have an internal diameter specification commonly expressed in inch units, and actually vary in range between about 0.230 inch and about 0.260 inch, even though 5 mm actually is the equivalent of 0.197 inches. In the present disclosure, therefore, “5 mm” or “about 5 mm”, when referring to the insertable profile of the device, or to the diameter of the shaft or the jaws in a closed configuration, refers to a diameter that is accommodated by presently available “5 mm” trocars. More particularly, embodiments of the shaft and closed jaws disclosed herein typically have a diameter in the range of about 0.215 inch to about 0.222 inch.

Embodiments of the electrosurgical device have an end effector such as a set of two opposing jaws or forceps that include one or more bipolar electrode pairs disposed on tissue engaging surfaces of the jaws, the device being adapted to effect tissue sealing and cutting. In some embodiments, the device includes a single bipolar electrode pair, one electrode in each of the jaws. In these embodiments, the electrodes are typically powered by a generator operating with a single radiofrequency channel. Other embodiments of the device may include a plurality of bipolar electrode pairs, and an operation by way of a plurality of radiofrequency channels. Some particular embodiments of the technology may take the form of non-electrical surgical device whose operation takes advantage of the mechanical and dimensional aspects of the technology. Some embodiments are useful in laparoscopic surgery and/or in non-laparoscopic surgery. In some embodiments, a standoff member is provided to maintain a physical gap between the pair(s) of electrodes.

In accordance with an aspect of the invention, an embodiment of an electrosurgical device comprises a jaw assembly structure which includes an upper jaw assembly, and a lower jaw assembly each having at least one electrode. At least one standoff member is provided on at least one of the upper jaw assembly and the lower jaw assembly. Thus, a direct contact between the opposing electrodes can be avoided.

In this electrosurgical device, the standoff member may be a single U-shaped standoff member provided on a jaw of the upper jaw assembly or the lower jaw assembly, so as to maintain a predetermined gap between upper and lower electrodes of the upper and lower jaw assembly, respectively. The U-shaped standoff member may be implemented as a unitary member.

In one or more embodiments, the standoff member may comprise a longitudinal slot provided down through its middle so as to allow a cutting element such as a knife to be advanced through a material such as a tissue grasped between the jaws.

In one or more embodiments, at least one, or both, of the upper jaw assembly and lower jaw assembly comprises a jaw arm, a carrier and electrodes wherein the carrier is provided with a longitudinal, optionally dove-tailed, ridge along its surface, and the jaw arm is provided with a mating, optionally dove-tailed, slot along its lower surface for receiving the ridge.

In one or more embodiments, at least one, or optionally two slots, which may optionally be dove-tailed, may be provided in a surface of the carrier for securing electrodes therein, wherein the electrodes may optionally have a dove-tail shape.

In one or more embodiments, the standoff member may be formed integrally with a center portion of the carrier, preferably the lower carrier.

In one or more embodiments, or according to another aspect of the invention independent of, or in any arbitrary combination with the above or below discussed further aspects of the invention, pivotable vertebrae may be provided for allowing the jaw assembly structure to articulate.

In one or more embodiments, a U-shaped region of the standoff may reside between upper electrodes and lower electrodes so as to keep the upper and lower electrodes a uniform distance apart of about 0.125 to 0.225 mm, or about 0.125 to 0.175 mm, or about 0.150 to 0.175 mm.

In one or more embodiments, at least one of the jaw assemblies may be provided with a central conductive body which is overmolded with a non-conductive portion including a raised lip.

In one or more embodiments, the raised lip may extend around the periphery of the conductive body and covers an edge portion of the face of the conductive body, the overmolded lip being configured to be interposed between an upper electrode or conductive body and a lower electrode or lower jaw when the upper and lower jaws are in a closed position.

In one or more embodiments, cross straps may be provided across portions of an electrode face.

In one or more embodiments, overmolded plugs may be connected with the cross straps which overmolded plugs pass through the central conductive body.

In one or more embodiments, peripheral standoff members may be provided over electrode edges with inwardly protruding fingers. Thus, a direct contact between the opposing electrodes can be avoided.

### BRIEF DESCRIPTION OF THE DRAWINGS

FIG. 1A is a perspective view of an embodiment of a laparoscopic electrosurgical device.

FIG. 1B is a side view of an embodiment of an electrosurgical device with the jaws in an open position.

FIG. 1C is a perspective view of an embodiment of an electrosurgical device with the jaws in a closed and locked position, and with the blade in a retracted in proximal position.

FIG. 1D is a perspective view of an electrosurgical device with the jaws in a closed and locked position, and with the blade in a distally advanced position.

FIG. 2A is a transparent perspective view of an embodiment set of jaws of an electrosurgical device, with the jaws in an open position.

FIG. 2B is a transparent perspective view of an embodiment of a lower jaw of a set of jaws an electrosurgical device, with a blade moved distally to a position about half way to its distal stop point.

FIG. 3A is a side view through the longitudinal midline of an embodiment of a set of jaws of an electrosurgical device, with the jaws in an open position.

FIG. 3B is a side view through the longitudinal midline of an embodiment of a set of jaws of an electrosurgical device, with the jaws in a closed position.

FIG. 3C is a side view through the longitudinal midline of an embodiment of a lower jaw of a set of jaws an electrosurgical device.

FIG. 4A is a side view through the longitudinal midline of an embodiment of a set of jaws of an electrosurgical device, with the jaws in an open position, and further showing a blade in a proximal and raised holding position.

FIG. 4B is a side view through the longitudinal midline of an embodiment of a set of jaws of an electrosurgical device, with the jaws in a closed position, and further showing a blade in a proximal and lowered holding position, ready to be distally advanced.

FIG. 4C is a side view through the longitudinal midline of an embodiment of a set of jaws of an electrosurgical device, with the jaws in a closed position, and further showing a blade in a distally advanced position.

FIG. 4D is a perspective view of a blade isolated from the shaft and jaws.

FIG. 5A is a perspective view of an alternative embodiment of an electrosurgical device with the jaws in an open position.

FIG. 5B is a side view of an embodiment of an alternative embodiment of an electrosurgical device with the jaws closed to a position where the distal tips of the jaws are in contact.

FIG. 5C is a side view of an embodiment of an alternative embodiment of an electrosurgical device with the jaws in a fully closed position.

FIG. 6 is a distal looking perspective view of an embodiment of a set of jaws of an electrosurgical device with the jaws in a closed position, a cross sectional exposure showing a passage through which a blade may be distally advanced.

FIG. 7A is a side view of an embodiment of set of jaws of an electrosurgical device, with the jaws in an open position.

FIG. 7B is a side view of an embodiment of set of jaws of an electrosurgical device, with the jaws at an initial point of closure, when the distal tips of the jaws have first made contact each other and a gap remains between the jaws at their proximal end.

FIG. 7C is a side view of an embodiment of set of jaws set of an electrosurgical device, with the jaws in a fully closed position, wherein the jaws are in full contact with each other from distal tip to proximal end.

FIG. 7D is a side view of a set of jaws of an embodiment of an electrosurgical device in a partially closed position, with the jaws as they would be positioned when closing around a portion of relatively thick target tissue, the jaws in a parallel alignment, spaced relatively widely apart by the presence of thick tissue therebetween.

FIG. 7E is a side view of a set of jaws of an embodiment of an electrosurgical device in a partially closed position, with the jaws as they would be when closing around a portion of relatively thin target tissue, the jaws in a parallel alignment, spaced apart by a narrow gap, reflecting the presence of thin tissue therebetween.

FIG. 8 is a perspective and upward looking view of a set of jaws of an embodiment of an electrosurgical device with the jaws in an open position, the view showing, more specifically, an isolated upper jaw, an isolated distal pivotable piece of a lower jaw, and an actuator wire looped around an attachment point at the proximal end of the upper jaw.

FIG. 9A is a side view of an embodiment of an isolated lower jaw of an electrosurgical device, the lower jaw including a proximal jaw piece that is fixed with respect to the shaft and a distal pivotable jaw piece mounted at a substantially central point of the distal piece on the proximal jaw piece.

FIG. 9B is a perspective and exploded view of an embodiment of a isolated lower jaw of a laparoscopic electrosurgical device, the lower jaw having a proximal jaw piece fixed to a shaft and distal pivotable jaw piece, the proximal and distal jaw pieces shown in an exploded relationship.

FIG. 9C is a bottom view of a lower jaw of an embodiment of an electrosurgical device, showing a connection between a proximal fixed jaw piece and distal pivotable jaw piece.

FIG. 9D is an upward looking perspective view of an embodiment of a distal piece of a lower jaw of an electrosurgical device.

FIG. 10A is a semitransparent side view of an embodiment of a lower jaw of an electrosurgical device, showing a proximal jaw piece and pivotably connected distal pivotable jaw piece, the distal pivotable piece in its default biased position, the distal end of the distal pivotable jaw piece pivoted to its upper end point, toward an upper jaw (not shown).

FIG. 10B is a semitransparent side view of an embodiment of a lower jaw of an electrosurgical device, showing a pivotably connected proximal jaw piece and distal pivotable jaw piece, the distal end of the distal pivotable jaw piece pivoted toward its lower end point, the proximal end of the distal pivotable jaw piece pivoted toward its upper end point, such a position putting the lower jaw in a substantially parallel relationship with the upper jaw (not shown).

FIG. 11A is a side view of an embodiment of a lower jaw of an electrosurgical device similar to the view shown in FIG. 10A, showing a leaf spring attached an upper aspect of the proximal jaw piece, the spring pushing against the distal pivotable jaw piece so as to maintain the distal pivotable piece in its default biased position, the distal end of the distal pivotable jaw piece pivoted to its upper end point.

FIG. 11B is a side view of an embodiment of a lower jaw of an electrosurgical device similar to the view shown in FIG. 10B, showing a leaf spring attached an upper aspect of the proximal jaw piece, the spring collapsed by the pressure being exerted on the distal end of the distal pivotable piece of the jaw, as would occur during closure of the jaw.

FIG. 12A is a proximal-looking perspective view of an embodiment of distal tips of a closed set of jaws of an electrosurgical device, the distal tips aligned by complementary longitudinal aligning features, a V-shaped projection on the lower jaw, and a V-shaped recession on the upper jaw.

FIG. 12B is a proximal-looking front view of an embodiment of the distal tips of a closed set of jaws of a laparoscopic electrosurgical device, the distal tips aligned by complementary longitudinal aligning features, a V-shaped projection on the lower jaw, and a V-shaped recession on the upper jaw.

FIG. 12C is a proximal-looking perspective view of a distal aspect of an electrosurgical device, with a set of jaws in an open position showing complementary longitudinal aligning features, a V-shaped projection on the lower jaw, and a V-shaped recession on the upper jaw, as well as a central longitudinally-oriented gap in both V-shaped surfaces that form a through passage for a blade that is distally advanceable when the jaws are in a closed position.

FIG. 13A is a proximal looking perspective view, partially exposed, of an embodiment of an electrosurgical device that shows aspects of the proximal portion of a set of jaws through which jaw actuator cables transit; the jaw actuator cables also serve as an electrical conduit to the upper jaw.

FIG. 13B is a proximal looking perspective view of an embodiment of an electrosurgical device that shows aspects of the proximal portion of a set of jaws through which jaw actuator cables transit.

FIG. 13C is a distal looking transparent perspective view of an embodiment of an electrosurgical device that shows aspects of the proximal portion of a set of jaws through which jaw actuator cables transit.

FIG. 13D is a distal looking transparent perspective view of an embodiment of an electrosurgical device similar to FIG. 13C, that shows aspects of the proximal portion of a set of jaws through which jaw actuator cables transit, with the cables in place.

FIG. 13E is a longitudinal section view, slightly offset from midline, showing the paths of cables through the distal portion of the shaft and into the proximal aspect of the jaws.

FIG. 13F is proximal looking perspective view of the proximal end of a lower jaw that is inserted into the distal end of a shaft, further showing engagement of the proximal end of the shaft with a cable isolator unit.

FIG. 14A is a bottom perspective view of an embodiment of an upper jaw of an electrosurgical device that shows plastic insulator layer overlaying the electrode.

FIG. 14B is a top perspective view of an embodiment of an upper jaw of an electrosurgical device that shows polymer insulator layer overlaying the electrode.

FIG. 14C is a top perspective view of an embodiment of an upper jaw of an electrosurgical device that shows polymer insulator layer overlaying the electrode, with the proximal portion of the jaw truncated to expose a cross section.

FIG. 15A is a top perspective view of an embodiment of an upper jaw of an electrosurgical device that shows points of ceramic overlaying the electrode at abrasive stress points.

FIG. 15B is a top perspective view of an embodiment of an upper jaw of an electrosurgical device that shows points of ceramic overlaying the electrode at abrasive stress points as they are embedded in a more extensive polymer layer.

FIG. 15C is a top perspective view of an embodiment of a pair of closed jaws of an electrosurgical device that shows points of ceramic overlaying the electrode at abrasive stress points as they are embedded in a more extensive polymer layer.

FIG. 16A is an exposed perspective view of a handle of an embodiment of an electrosurgical device that shows aspects of the proximal end of a rotatable shaft.

FIG. 16B is a perspective view of an isolated proximal end of a rotatable shaft.

FIG. 16C is a midline sectional view of an isolated proximal end of a rotatable shaft.

FIG. 16D is a midline sectional view of a proximal portion of a rotatable shaft.

FIGS. 17-23 are various views of an additional embodiment having a single, unitary standoff member between electrodes.

FIGS. 24-32 are various views showing further embodiments having single standoff members.

### DETAILED DESCRIPTION

Embodiments of the technology described herein provide various improvements over available electrosurgical devices, such improvements permitting a physical downsizing of a device to a dimension that permits practical use of an electrosurgical device within the constraints of a laparoscopic surgical environment. One of these constraints to working laparoscopically relates to the 5 mm inner diameter opening provided by a commercially standard trocar. A device compatible with the 5 mm opening constraint needs to have an insertable configuration with a maximal diameter that is insertable therethrough. These technological improvements are generally directed toward creating a high degree of efficiency with regard to performance of the device per unit volume or cross sectional area. For example, a jaw set of a disclosed device, in spite of small physical dimension, is able to deliver an appropriate level of force to tissue being clamped by the jaws, and the structure and material of the jaws have sufficient strength to maintain integrity during the delivery of such force.

In one aspect, the technology includes maximizing the amount of structural material in particular areas as a percent of total amount of device material. The proximal aspect of the jaw set, for example, includes various components, some that contribute structural support for the jaws, and other components that perform other functions, such as mechanical or electrical functions. The technology, in this aspect, is directed toward minimizing cross sectional area or volume that does not directly support the jaws. Some components of conventional electrosurgical devices are typically dedicated to a single use, such as electrodes, power lines, or actuator lines; in contrast, various components of embodiments of the presently disclosed device do double duty both as structural and electrical components in embodiments of the technology. In another example of material and occupied volume efficiency, some structural components, such as a pin connecting two jaws at their base, are eliminated and replaced by a pinless mechanism that links upper and lower jaws of a jaw set together.

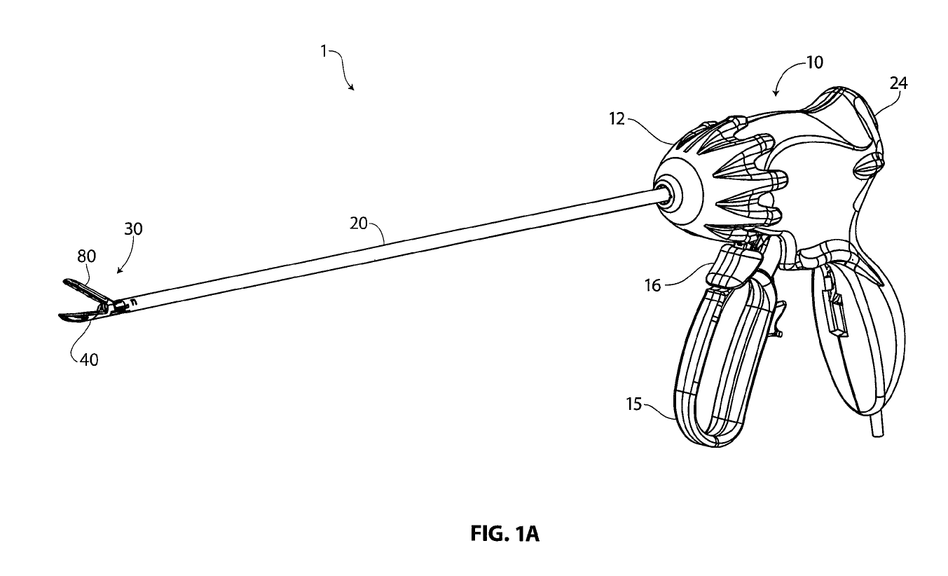

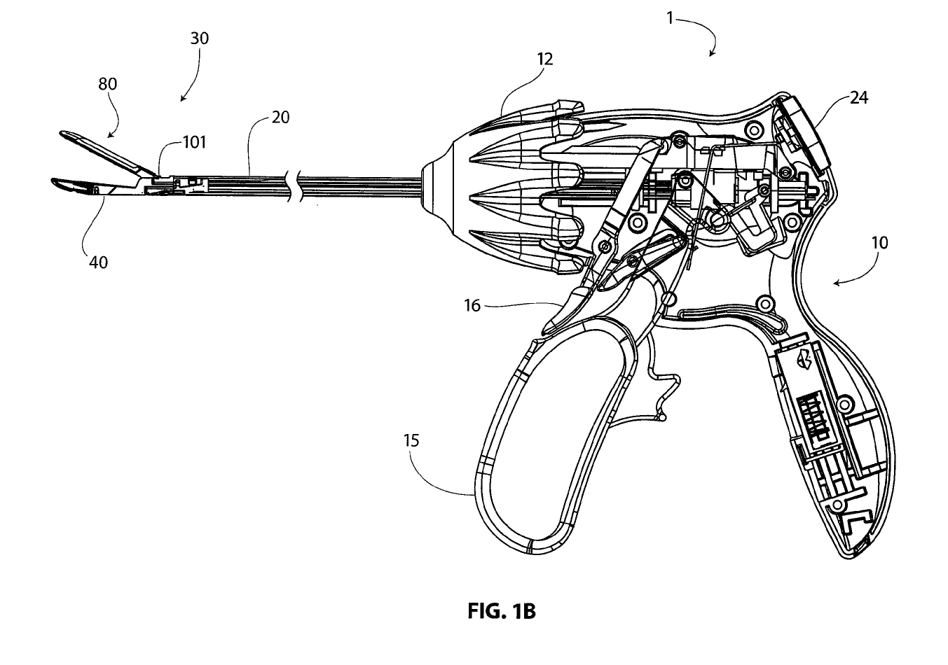

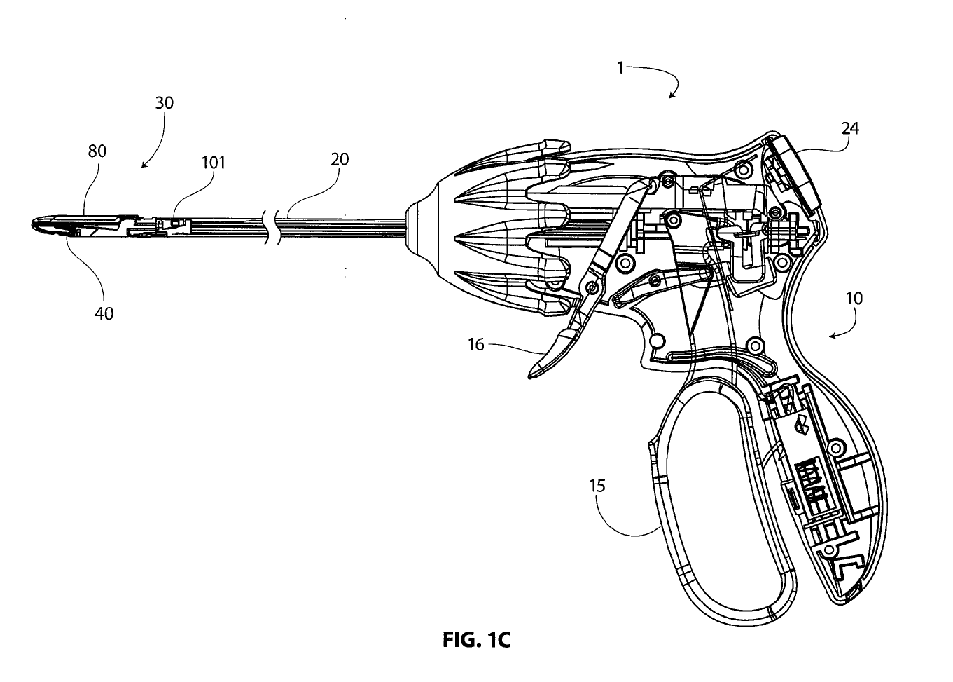

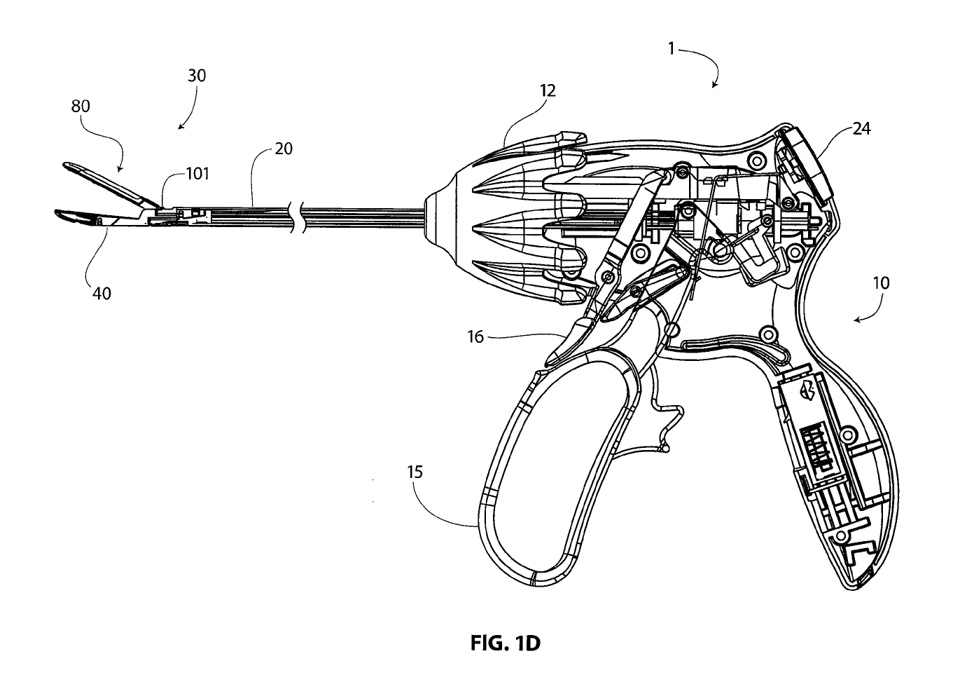

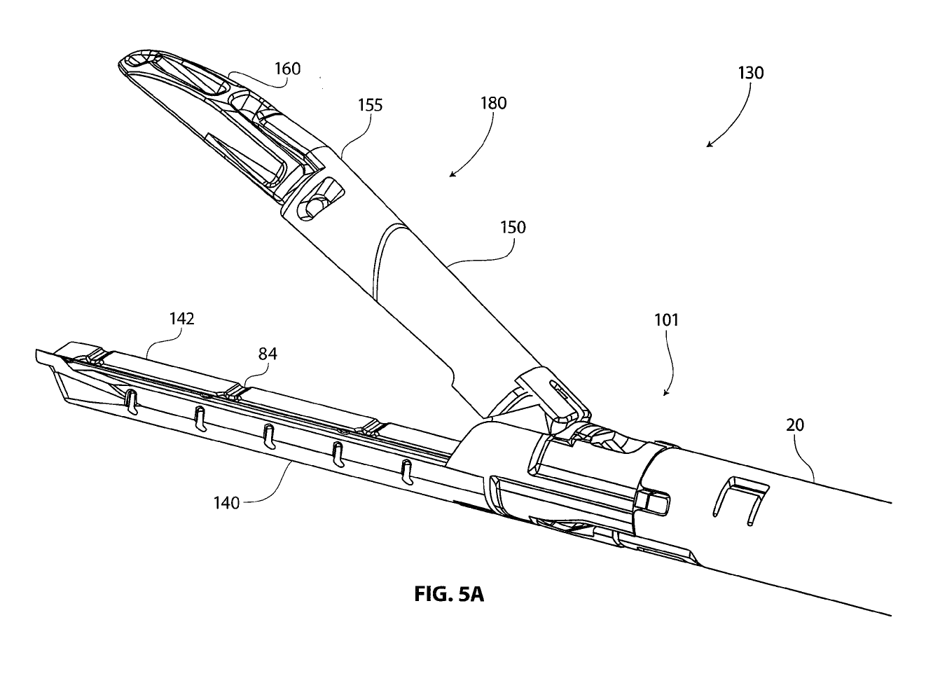

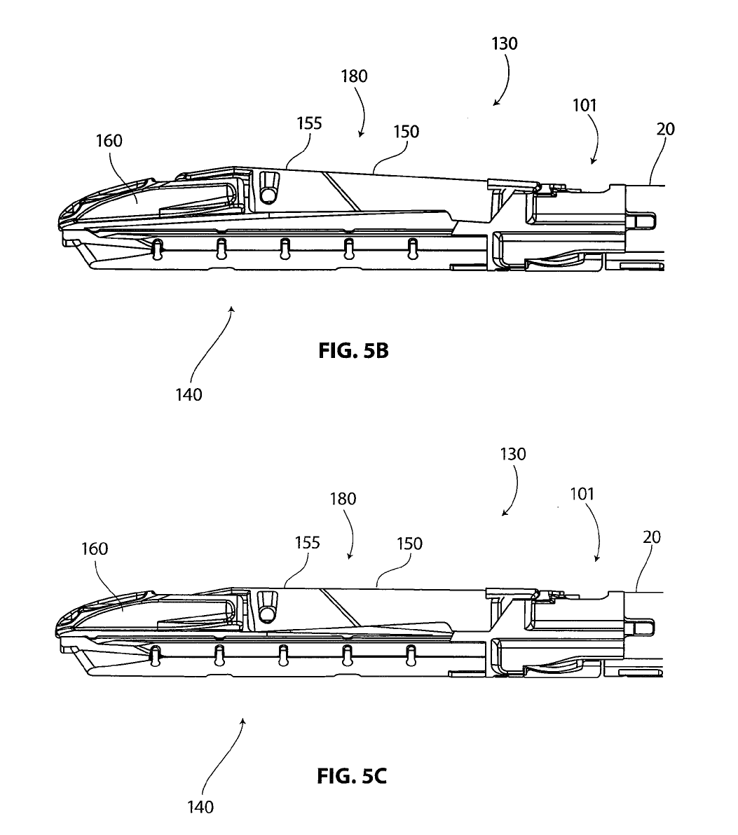

Aspects of the technology in the form of embodiments of the disclosed electrosurgical device and methods of using the device are illustrated in FIGS. 1-16D. With regard to Embodiments A and B, as described above, the majority of the figures depict examples of Embodiment A, or they relate to aspects of the technology that are common to both Embodiments A and B. FIGS. 5A-5C particularly depict examples in accordance with Embodiment B. It should be understood that in any reference to a lower jaw or an upper jaw when describing the figures is for a convenient visual reference with respect to a conventional positioning of the rotatable jaws, and that the two jaws could be more generally referred to as a first jaw and a second jaw. Further, with respect to orientation of the figures, in general a distal end of a device is on the left, and a proximal end of a device is on the right.

FIGS. 1A-1D provide various views of embodiments of a laparoscopic electrosurgical device as a whole. FIG. 1A is a perspective view of an embodiment of an electrosurgical device 1 as provided herein, with a set of jaws 30 in an open position. FIG. 1B is a side view of an embodiment of an electrosurgical device 1 with the jaws 30 in the same open position as in FIG. 1A. A handle 10 supports a jaw actuator grip 15 and blade actuator lever 16, and a shaft rotator 12. A shaft 20 extends distally from the handle, and supports an end effector such as a set of jaws 30 at its distal end. In the embodiments described and depicted herein, the end effector takes the form of a forceps or pair of jaws 30, with a first law or lower jaw 40 and a second jaw or upper jaw 80. A pinless rotation assembly or mechanism 101 operates pivoting of the jaws between an open position and a closed position.

The shaft rotator 12 is configured to move freely in both clockwise and counterclockwise directions, and in so moving, rotates the shaft around its longitudinal axis. Rotation of the shaft translates into rotation of the end effector 30 around its longitudinal axis. The jaw actuator grip 15 is operably connected to end effector 30 by an actuation wire disposed within the shaft, which is configured to open and close the jaws. The actuation wire is configured as a push and pull mechanism, where in a push of the wire opens the jaws and a pull on the wire closes them. A biasing mechanism within the handle at the proximal end of the wire maintains a distal-ward bias that pushes the wire, maintaining the jaws in a default open position. A proximal pull on the jaw actuator grip 15 pulls the actuator wire proximally, causing the jaws to pull. The jaw actuator grip is lockable in its proximally pulled position, thereby locking the jaws in a closed position. A second pull on the jaw actuator grip releases the lock, thereby allowing the jaws to open. The blade actuation lever 16, positioned in this embodiment distal to the jaw actuator grip, is connected by mechanical linkage to a blade disposed within the shaft. A pull on the blade actuation lever moves the blade forward distally, to effect a separation of tissue after it has been sealed by radiofrequency energy delivered to the tissue by bipolar electrodes within the set of jaws. A radiofrequency on/off button 24 is positioned at an upper proximal site on the handle.

FIG. 1C is a perspective view of an embodiment of an electrosurgical device 1 with the jaws 30 in a closed and locked position, and with the blade in a retracted in proximal position. FIG. 1D is a perspective view of an electrosurgical device 1 with the jaws 30 in a closed and locked position, and with the blade in a distally advanced position. The blade itself, is not visible in these figures, but the forward position of the depicted blade actuator lever 16 in FIG. 1C is indicative of the blade being in a retracted or home position, and the pulled back position of the blade actuator lever in FIG. 1D is indicative of the blade being in a forward position. FIG. 1C also shows the jaw actuator grip in a pulled back position, locked into the main handle piece 10. In this position, and typically only in this position, is the blade actuator lever free to be pulled back so as to advance the blade distally.

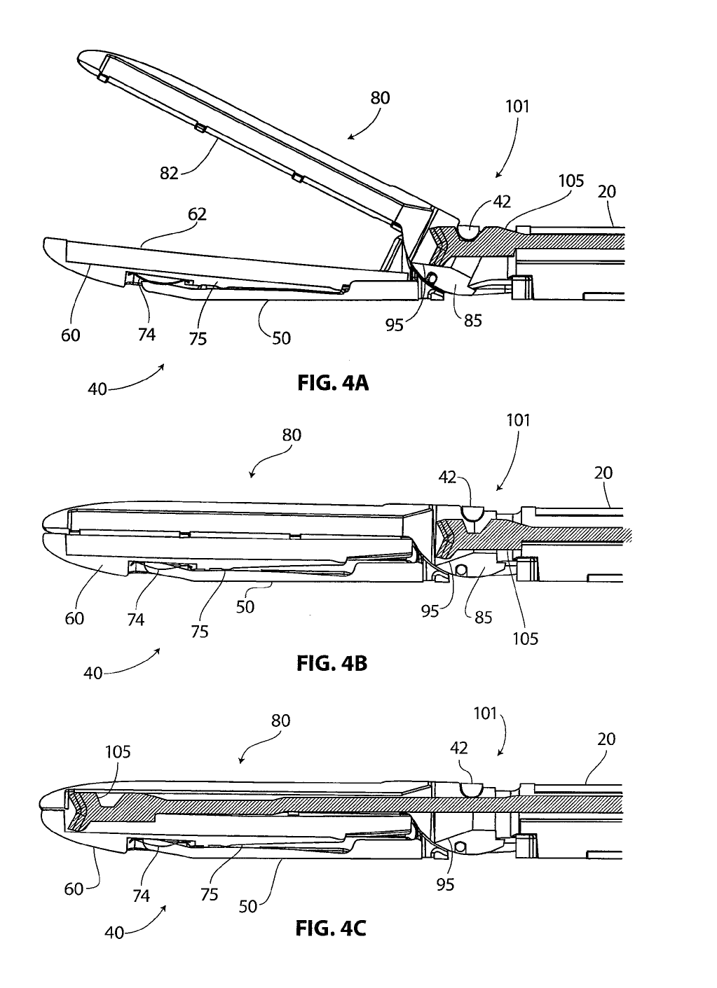

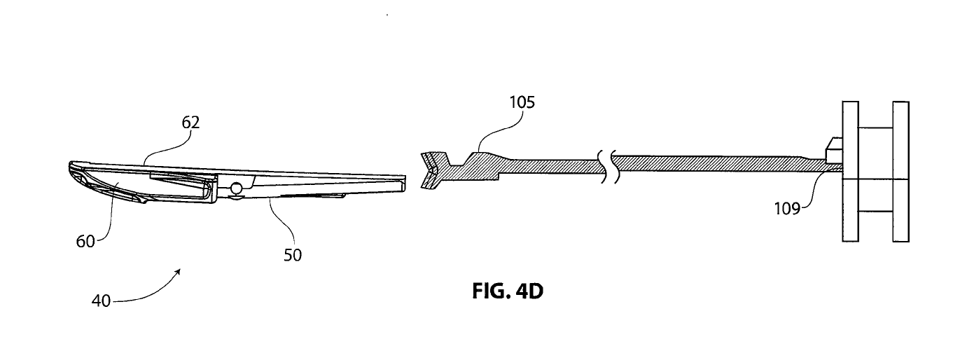

Embodiments of electrosurgical devices, as described herein, may be configured such that the (1) provision of radiofrequency energy delivery to seal tissue portions and (2) the movement of the blade to sever or separate sealed tissue portions are separate and independent operations. Distal movement of the blade from its proximal home position is typically allowed only when the jaws are closed and in a locked position, the locking occurring by way of engagement between the jaw actuator grip and elements within the handle. (As described further below, in the context of describing FIG. 4A, a jaw-based blocking system also operates to prevent distal movement of the blade when the jaws are closed.) Once the jaws are in such a locked position, the blade is free to move through its full range of proximal to distal movement. Although the blade is free to move when the jaws are closed and locked, its default and biased position is its proximal home position; pressure from blade actuator lever 16 needs to be maintained in order for the blade to remain at its most distal position. Further detail related to the distal movement of the blade is provided below in the context of FIGS. 4A-4D.

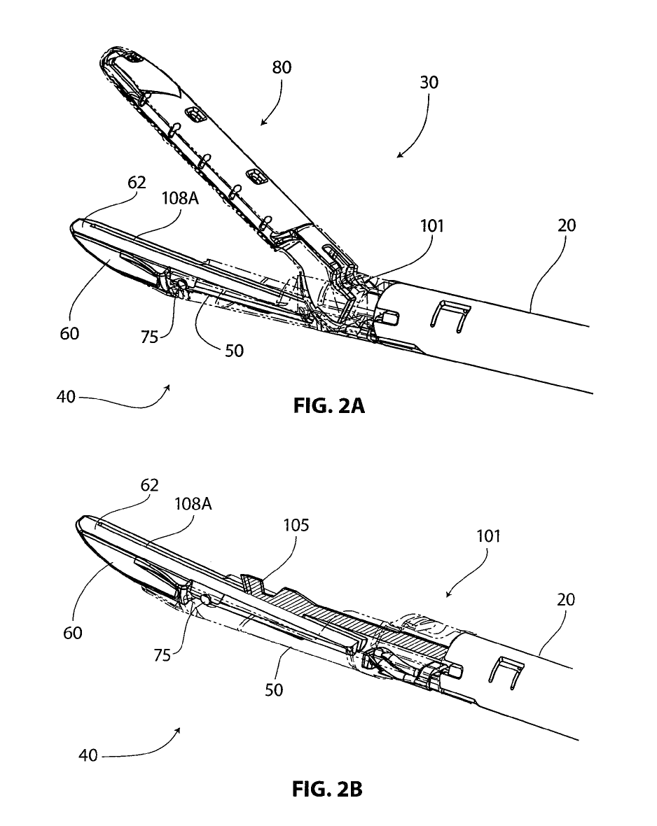

FIGS. 2A and 2B provide similar transparent views of embodiments of a set of jaws 30 in an open position; these figures show a pinless rotation mechanism or assembly 101 that comprises proximal aspects of both the lower jaw 40 and the upper jaw 80. FIG. 2A is a transparent perspective view of a set of jaws of laparoscopic electrosurgical device in an open position, with a blade 105 disposed in a proximal or home position within a proximal space in the jaws, and extending further into a distal portion of the shaft. FIG. 2B is a transparent perspective view of a lower jaw of set of jaws of laparoscopic electrosurgical device with a blade moved distally to a position about half way to its distal stop point.

An embodiment of a pinless rotation assembly 101, as shown in FIGS. 2A and 2B includes a first arcuate track portion 85 of upper jaw 80 and a second arcuate track portion 45 of lower jaw 40. Aside from the specific structures that comprise rotation assembly, identifier 101 in figures generally designates a junctional region of the devise that includes the proximal aspects of both upper and lower jaws. Because of the transparency of the drawing, arcuate track 45 of lower jaw 40 is difficult to see; it is shown in greater solid detail in further figures. Arcuate track 85 of upper jaw 80 is rendered as a solid. Further visible in these figures is the surface of an electrode tray or bipolar electrode 62, within the pivotable portion 60 of lower jaw 40. Blade track or passageway 108A is centrally disposed within electrode 62. A companion facing half of the full blade track is similarly disposed (not visible) within the electrode portion of upper jaw 80.

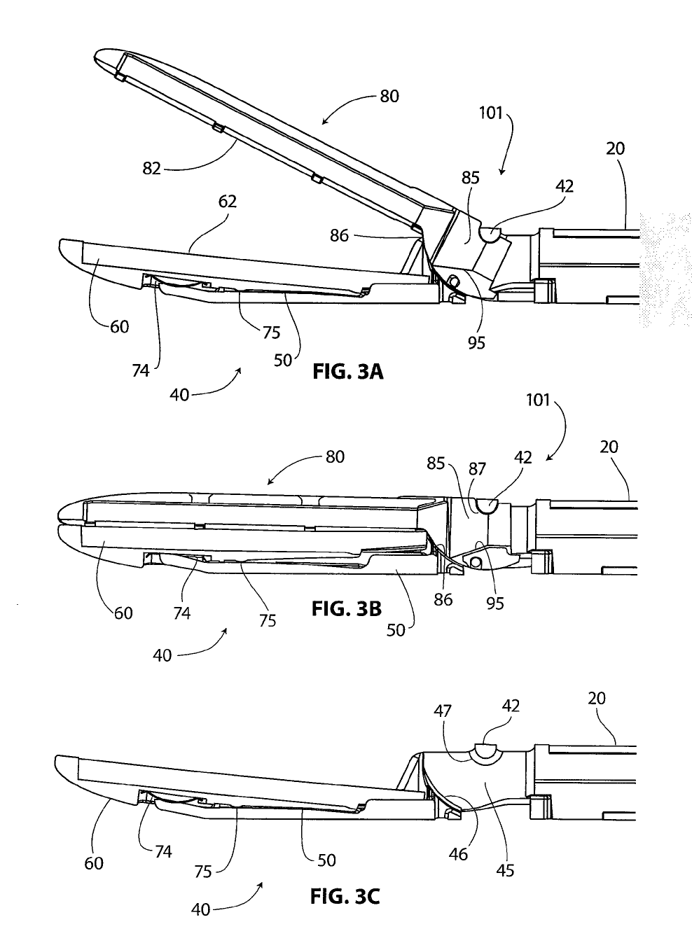

FIGS. 3A-3C provide a side views through the longitudinal midline of an embodiment of a set of jaws of a laparoscopic electrosurgical device; the blade is not shown in these views. FIG. 3A shows the jaws in an open position; FIG. 3B shows the jaws in a closed position. FIG. 3C shows the lower jaw 40 in isolation, without the upper jaw. FIGS. 3A-3C collectively focus on an embodiment of a pinless rotation assembly 101 that joins upper jaw 80 and lower jaw 40 together, and allows the jaws to pivot with respect to each other. More specifically, pinless rotation assembly 101 allows the upper jaw to pivot with respect to the proximal base portion 50 of lower jaw 40. Notably, the rotation assembly does not include a through pin. More particularly, these figures focus on arcuate track portions of both jaws that cooperate to allow the jaws to open and close. A first arcuate track 45 is formed on a proximal aspect of a proximal portion 50 of lower jaw 40. A second arcuate track 85 is formed on a proximal aspect of upper jaw 80. FIG. 3C shows the lower jaw 40 in isolation unimpeded by the intervening appearance of upper jaw, and provides the best view of a first arcuate track 45, with its upper and smaller concentric surface 47 and lower and larger concentric surface 46.

Both of the first and second arcuate tracks include concentric surfaces, one surface smaller and more central to the other, and the other surface larger and more peripheral to the other. First arcuate track 45 of lower jaw 40 (more particularly of proximal portion 50 of lower jaw 40) has a larger concentric engagement surface 46 on its lower aspect, and it has a smaller concentric surface 47 on its upper aspect. Second arcuate track 85 of upper jaw 80 has a larger concentric engagement surface 86 on its lower aspect, and it has a smaller concentric surface 87 on its upper aspect. As a whole, second arcuate track 85 (of upper jaw 80) is generally contained within an enclosure provided by first arcuate track 45 (of lower jaw 40). The first and second arcuate tracks are dimensioned such that the second arcuate track can freely rotate within first arcuate track. The two larger concentric surfaces, i.e., the lower surface 46 of the lower jaw and the lower surface 86 of the upper jaw are complementary. And the two smaller concentric surfaces, i.e., the upper surface 47 of the lower jaw and the upper surface 87 of the upper jaw are complementary.

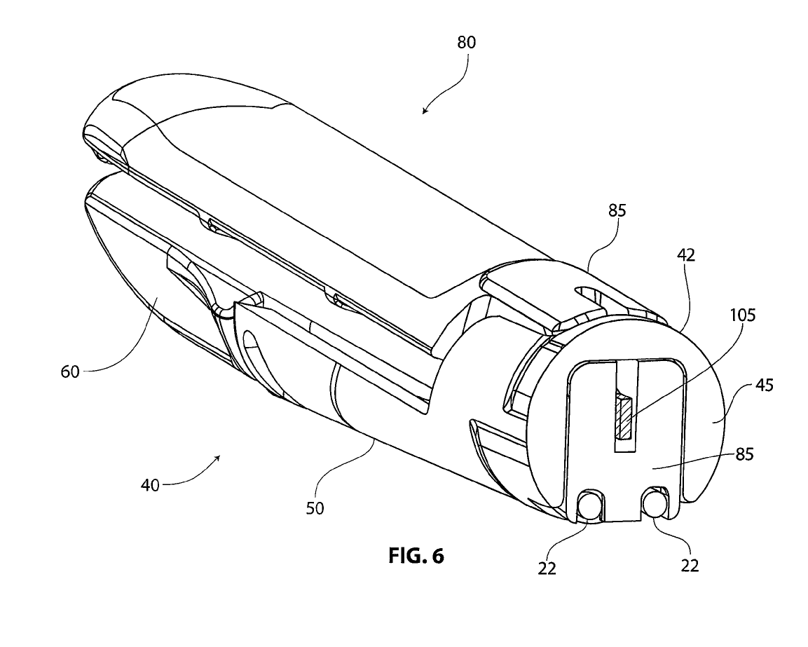

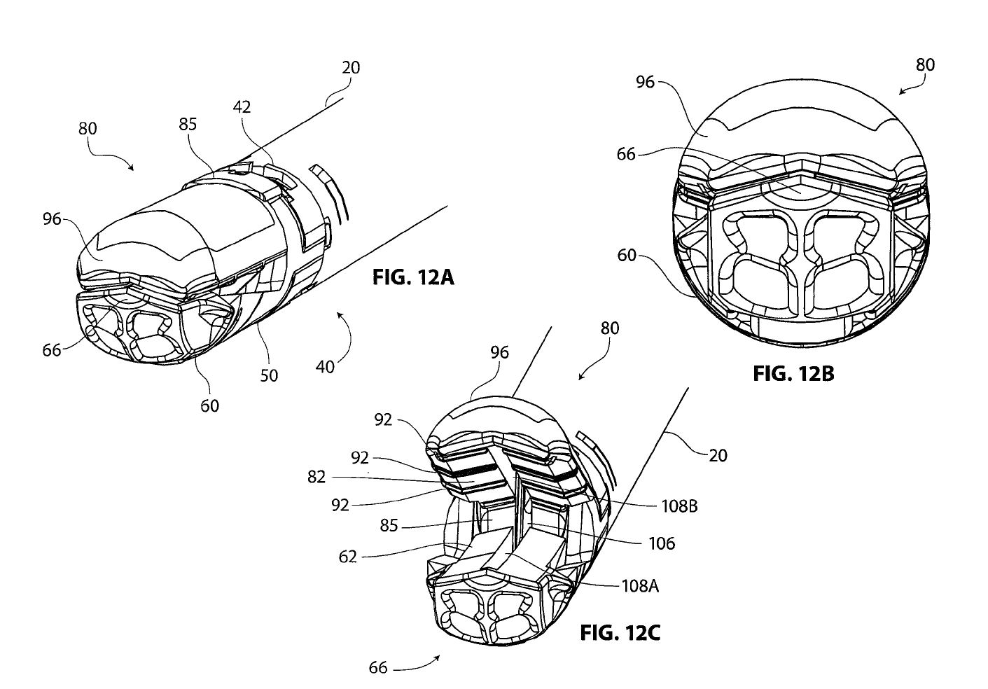

A detail of both first and second arcuate tracks, not seen in FIGS. 3A-3C since they are side views, is that they arcuate track includes a central slot to accommodate through passage of a blade 105. Aspects of the arcuate tracks and the blade through path may be seen in FIGS. 6 and 12 and will be described further below. The arrangement of complementary surfaces, and the enclosure of the second arcuate track within the first arcuate track permit the pivoting of the upper jaw 80 with respect to lower jaw 40. A retaining strap 42 of the proximal portion 50 of lower jaw 40 is arranged laterally across the top of the upper and smaller concentric surface 87. Retaining strap 42 securely retains the second arcuate track within the first arcuate track such that it cannot be lifted from within its enclosure.

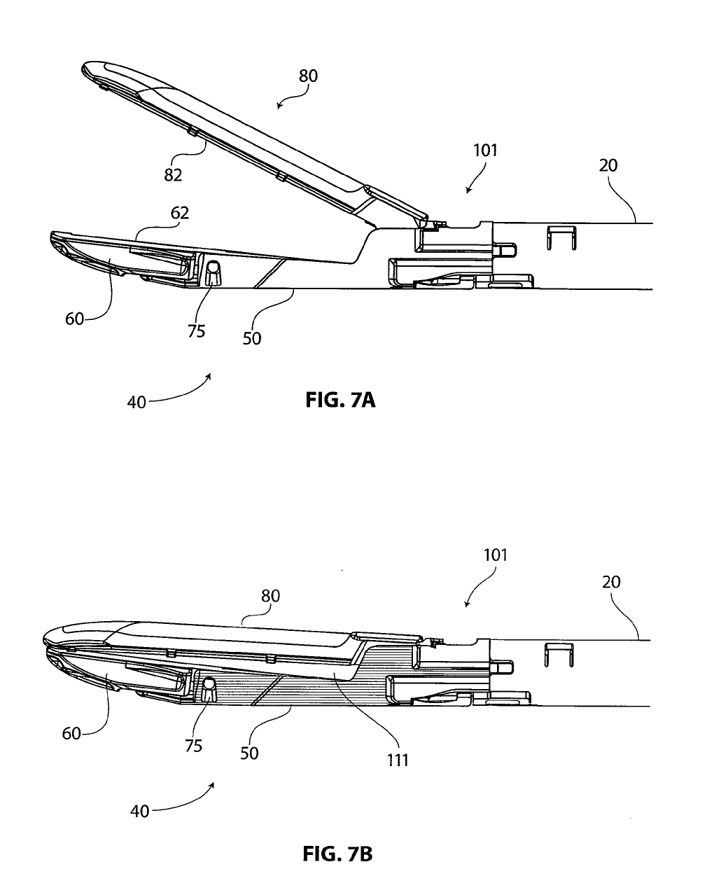

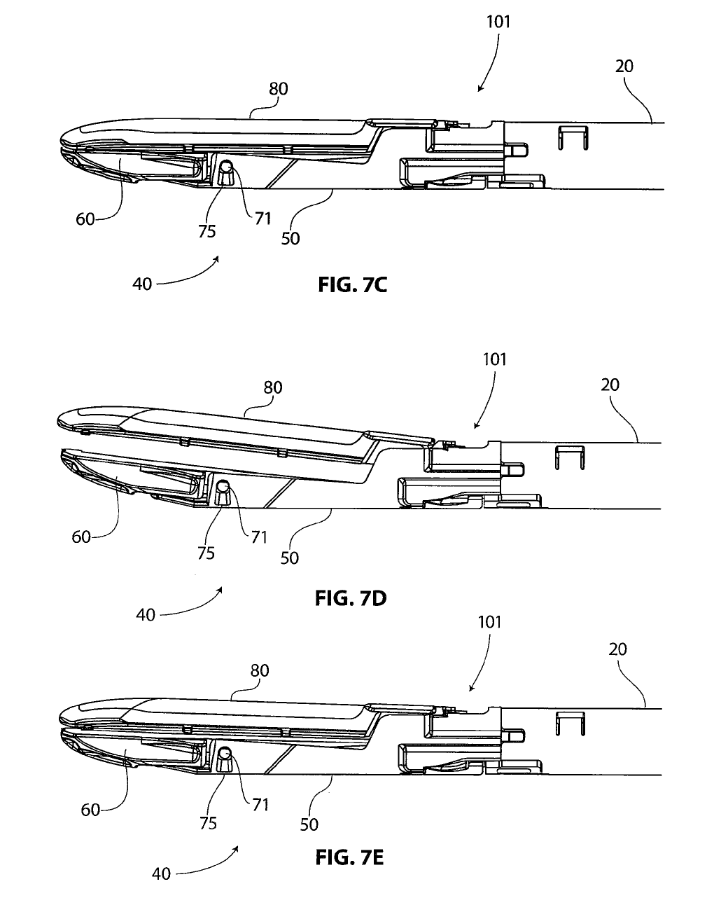

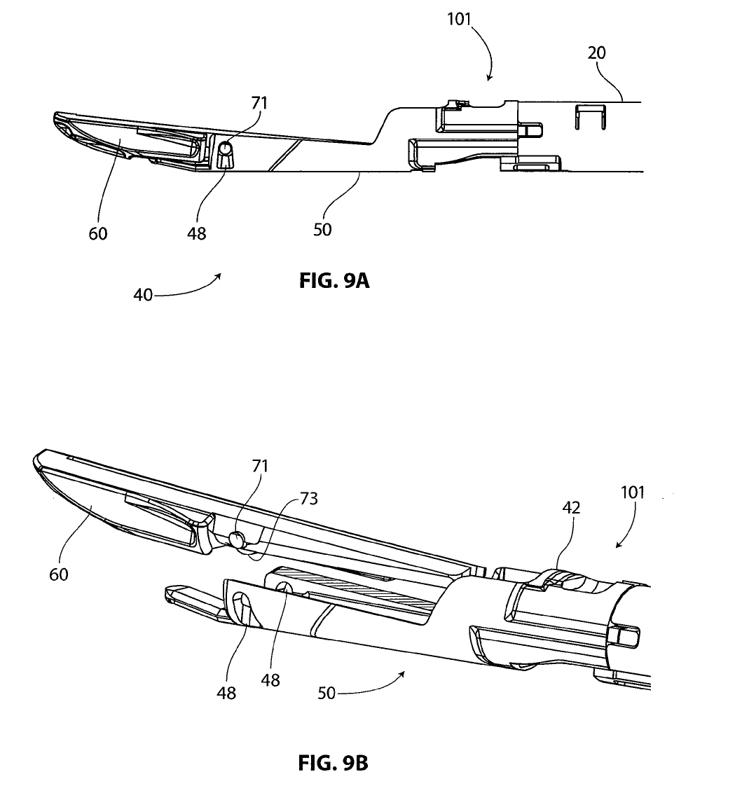

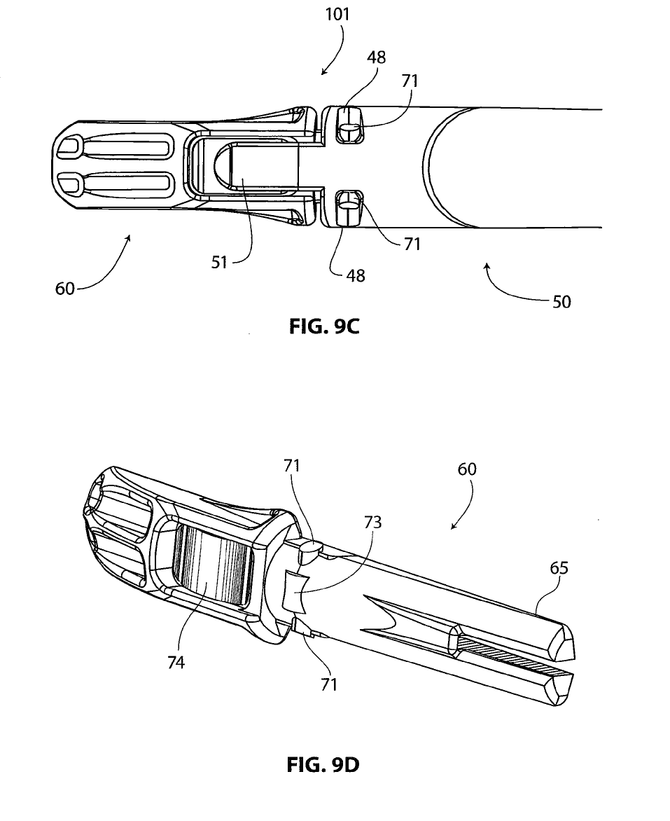

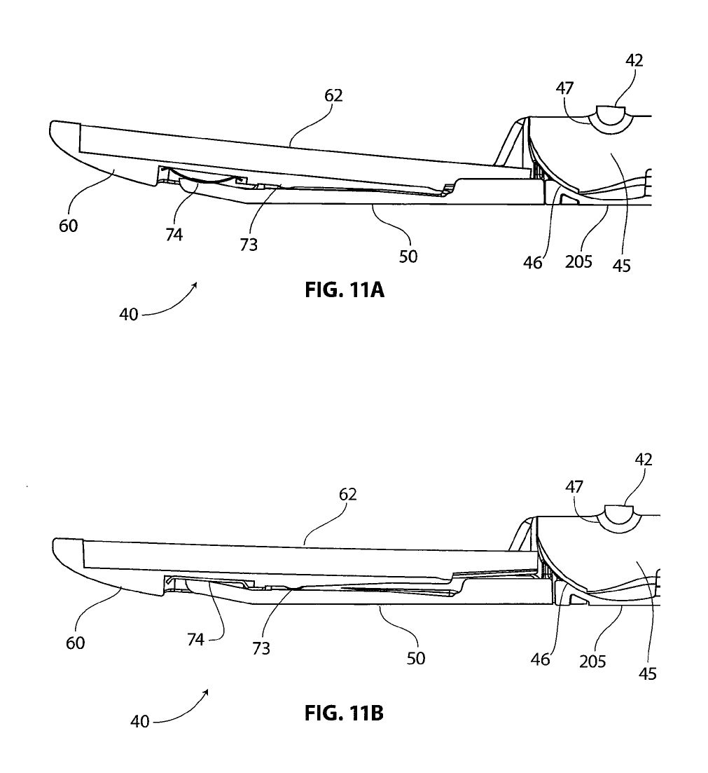

Also shown in FIGS. 3A-3C is the site of a pivotable connection 75 between distal jaw piece 60 and proximal jaw piece 50; aspects of pivotable connection 75 are described below in the context of FIGS. 7A-7C. Further shown in FIGS. 3A-3C is a biasing member 74, which is described below in the context of FIG. 9D and FIGS. 11A-11B.

FIGS. 4A-4D provide side views through the longitudinal midline of an embodiment of set of jaws and various views of an embodiment of a tissue dissecting blade, per the disclosed technology. The focus of these figures relates to aspects of the blade and its proximal holding space that prevents distal movement of the blade when the jaws are in an open position. FIG. 4A shows the device embodiment in an open position with a blade 105 in a proximal and raised holding position. FIG. 4B shows the device embodiment in closed position, with the blade 105 in a proximal and lowered holding position, ready to be distally advanced. FIG. 4C shows the device in closed position, with the blade in a distally advanced position. When blade 105 is in a proximal holding position, its bottom edge 105B rests on shelf 95, a feature of second arcuate track piece 85 of upper jaw 80. (Shelf 95 can also be seen in FIGS. 3A and 3B.) In comparing the views of FIG. 4A (jaws open) and FIG. 4B (jaws closed), it can be seen that when the jaws are open, shelf 95 is rotated to a raised position, and when the jaws are closed, shelf 95 is rotated to a lower position. The raised position of the shelf prevents distal movement of the blade; the lowered position of the shelf allows distal movement of the blade. FIG. 4D is a perspective view of a blade isolated from the shaft and jaws. At its proximal end, blade 105 is connected to a site 109 in the handle that is supported by a mechanical linkage that maintains the blade in a withdrawn or proximally biased position.

The pivoting of upper jaw 80 pivots upward so as to move jaw set into an open position is driven by the rotation of second arcuate track 85 within the enclosure of first arcuate track 45. As seen in FIG. 4A, as arcuate track 85 rotates upward (clockwise, in this view), its shelf 95 also rotates upward, lifting blade 105 upward. As blade 105 is lifted, its upper edge is lifted above the ceiling of distal ward opening of blade track or through passage 106. Blade track 106 is visible in the side views of FIGS. 4A and 4C, and can also be seen in FIG. 5A. When upper jaw 80 is closed with respect to lower jaw 40 (as in FIG. 4B), second arcuate track 85 and its blade shelf 95 is rotated downward, allowing blade 105 to drop into a position such that it has a clear path into blade track 106. This described and depicted relationship among the blade, the shelf of the rotatable second arcuate track (of upper jaw 80), and the blade track, thus creates a mechanism that prevents distal movement of the blade when the jaws are in an open position, allowing distal movement only when the jaws are in a closed position, as seen in FIG. 4C.

FIGS. 5A-5C provide views of an alternative embodiment (Embodiment B) of a laparoscopic electrosurgical device in which a set of jaws 130 includes a first jaw 140 that is unitary and fixed with respect to the shaft and the second jaw 180 is a two-piece jaw that is pivotable with respect to the shaft. More particularly, the two-piece (second) jaw of this embodiment has a proximal piece 150 that is pivotable with respect to the shaft, a distal jaw piece 160 that is pivotable with respect to the proximal piece, and a pivotable assembly 155 connecting the proximal jaw piece and the distal jaw piece. FIG. 5A provides a perspective view of this device embodiment with the jaws in an open position. FIG. 5B provides a side view of the embodiment with the jaws closed to a point where the distal tips of the jaws are in contact. FIG. 5C provides a side view of the embodiment with the jaws in a fully closed position. FIG. 5A shows the jaws without a polymer coating; this affords a view of troughs 84 within the electrode surface 142. Similar troughs are present in the upper jaw of embodiment A.

Other than the variation in the configuration of the jaws as just described, other aspects of embodiments A and B are substantially the same. In particular, the dynamics of the closing of the jaws of Embodiment B are the substantially the same as those of Embodiment A, which are described in detail below, in the context of FIGS. 7A-7E.

FIG. 6 provide distal looking perspective views of a set of jaws of an embodiment of laparoscopic electrosurgical device in closed position, more particularly, a cross sectional exposure shows a blade passage way or track 106 through which a blade may be distally advanced. The cross sectional slice on the right side of FIG. 6 reveals a section through first arcuate track 45 (of the proximal portion 50 of lower jaw 40) that substantially encloses second arcuate track 85 (of upper jaw 80). A proximal cross sectional slice through of blade 105 can be seen within slot 88 of second arcuate track 85. Slot 88 is contiguous with blade track 106 of the jaws, as seen best in FIG. 12C.

FIG. 6 also provides a view that allows a calculation of the proportion of the total cross sectional area of a critical portion of the device that provides forward supporting structure to the jaws. This portion of the device is a relevant site to consider for its structural content in that it includes the pinless rotational mechanism whereby the jaws pivot with respect to each other. In an otherwise more conventional structure, this area might include through pins or other structures that do not convey structural support to the jaws. In this area, thus, embodiments of a pinless rotation mechanism provide structural material content that might otherwise be missing. If a diameter of 0.218 inch is considered, which is consistent with the contiguous circular aspect of the base of the jaws is drawn, the cross sectional area included therein is about 0.0373 square inches. Through this section the cross sectional area of the upper jaw is about 0.0151 square inches, and that of the lower jaw is about 0.0155 square inches. The summed area of the upper and lower jaws is about 0.0306 square inches, or about 82% of the total cross sectional area.

FIGS. 7A-7E provide side views of a set of jaws of an embodiment of a laparoscopic electrosurgical device in an open position, and in several states of partial or initial closure and full closure. These figures focus on the pivotable relationship between distal pivotable piece or portion 60 and fixed proximal or base piece 50 of lower jaw 40, as enabled by pivotable rotation assembly or mechanism 75. The pivotable relationship between pivotable portion 60 and base portion 50 plays out in various ways that lower jaw 40 and upper jaw 80 approach each other as they close, particularly as they close around a portion of target tissue to be treated electrosurgically.

FIG. 7A shows the jaw embodiments in an open position. Pivotable jaw portion 60 of first jaw or lower jaw 40 is pivotable within its longitudinal axis at pivotable connection 75 through an arc with total rotational range of about 6 degrees. In various embodiments, the rotational range may be between about 2 degrees and about 8 degrees or more. In the open position as shown in FIG. 7A, pivotable jaw piece 60 is pivoted to its maximal degree of clockwise rotation, with the distal end of the pivotable jaw piece in a raised position. (The terms clockwise and counter clockwise are used in relative to the side view depicted, with the distal end of the jaw on the left hand side of the image.) This clockwise position is a default or biased position as shown in FIG. 11A, which show the lower jaw 40 isolated from upper jaw 80. This default position may be maintained by a push from a spring or biasing mechanism disposed at the proximal end of an actuator wire (not shown).

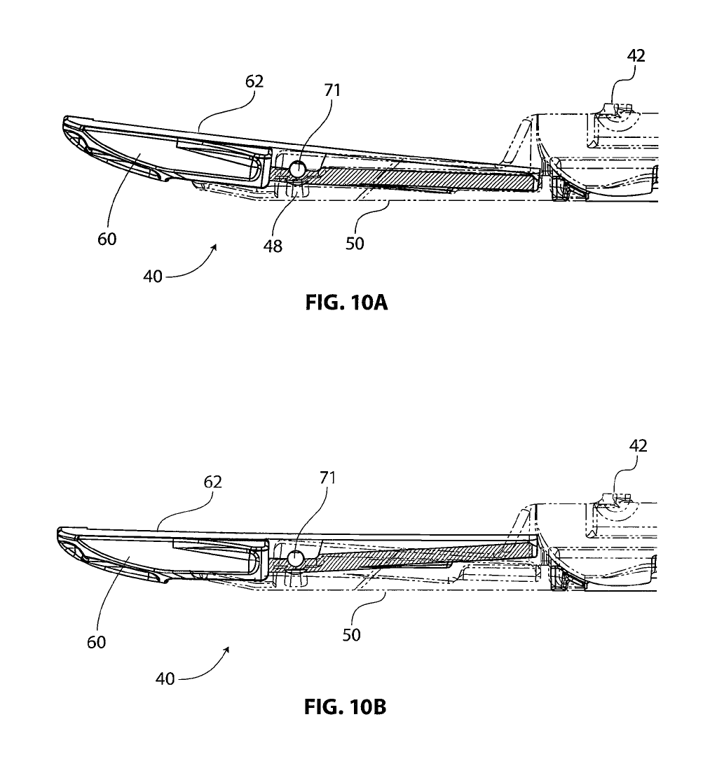

A clockwise rotation or pivoting of pivotable jaw piece 60 (of lower jaw 40) results in its distal end or tip 66 assuming a relatively high profile and its proximal aspect assuming a relatively low profile with respect to proximal jaw piece 50. The differences in profile are relatively subtle, but are apparent when the proximal aspect of the upper profile of the surface of electrode 62 is viewed in relationship to the upper surface of the proximal aspect of the proximal jaw piece 50. In FIG. 7A, for example, there is a relatively small linear profile of electrode 62 visible over the base provided by proximal jaw piece 50. The height of this profile, indicative of the relative degree of pivoting of the pivotable jaw piece 60, will be pointed out in the descriptions associated with FIGS. 7B-7E, below. The relationship between the pivoting of the pivotable jaw piece 60 with respect to base jaw piece 50 is also apparent in FIGS. 10A and 10B.

FIG. 7B shows an embodiment of a set of jaws at a point when they are moving toward a closed position, when the distal tips of the jaws (distal tip 96 of upper jaw 80 and distal tip 66 of lower jaw piece 60) first contact each other. Upon first contact of the tips of the jaws, a gap remains in the region between the jaws 111 at their proximal end. As in FIG. 7A, the pivotable piece 60 is in its default biased position, pivoted to its maximal degree of clockwise rotation. In this position, upon first contact of the tips, no pressure has yet been applied to the tips of the jaws. As in FIG. 7A, there is a relatively small linear profile of electrode 62 visible over the base provided by proximal jaw piece 50.

FIG. 7C shows the jaw embodiments in a fully closed position, with the jaws, from distal tip to proximal end, in full contact with each other. This relative positioning of the jaws may be understood as one that would occur when the jaws are being closed without intervening tissue between them, or when intervening tissue is very thin. Thus, this relative configuration is similar to that arrived at when the jaws are closed around a thin piece of tissue, as seen in FIG. 7E (described below), but without the intervening space occupied by tissue. This position is arrived at by a counter clockwise pivoting of the pivotable piece 60 of lower jaw 40 around pivotable connection 75 such that the distal tip of the pivotable piece has moved downward, and the proximal end of the pivotable piece has moved upward. Consistent with this raised aspect of the proximal piece of pivotable jaw piece 60, and in contrast to the view seen in FIGS. 7A and 7B, FIG. 7C shows there to be a relatively high linear profile of electrode 62 visible over the base provided by proximal jaw piece 50. Details of pivotable connection 75, in its components that are associated with both the pivotable jaw piece 60 and the distal base jaw piece 50 may be seen in FIGS. 9A-9D.

FIG. 7D shows the jaw embodiments in a partially closed position, with the jaws as they would be when closing around a portion of relatively thick portion of target tissue (not shown), but of a thickness that does not exceed the effective capacity of the jaws. The intra-jaw pivotability, as represented by first jaw 40, provides a capability for a set of jaws to align in a parallel or substantially parallel configuration as they close around a portion of tissue, a capability that provides an advantage over a set of conventional jaws without such intra-jaw pivotability. The configuration of jaws as depicted in FIG. 7D is one in which thickness of target tissue would likely exceed the therapeutically acceptable limit of thickness for a conventional set of jaws, but which is well within the therapeutically effective capacity.

A non-parallel closure of jaws, as is typical of conventional jaws that do not have intra-jaw pivotability or another compensatory mechanism, can have therapeutically unsatisfactory consequences, such as uneven distribution of pressure on tissue along the line of jaw contact, as well as uneven distribution of radiofrequency energy when delivered by electrodes. Embodiments of a set of jaws as provided herein, however, can of course still be confronted with a portion of target of tissue that exceeds their capacity for parallel closure of tissue engaging surfaces of jaws. However, as noted, the thickness of tissue that would account for the configuration of the jaws as seen in FIG. 7D is one that demonstrates the therapeutic advantage of the intra-jaw pivotability of lower jaw 40.

This relative positioning of the jaw embodiments as seen in FIG. 7D comes about for at least two reasons. First, the jaws are not completely closed at the level of the rotational assembly connecting the proximal aspects of the jaws. Second, as in FIG. 7C, this position has been arrived at by a counter clockwise pivoting of the pivotable piece 60 of lower jaw 40 around pivotable connection 75 at least partially through its range of angular rotation. From the default position of pivotable piece 60, this clockwise rotation has moved the distal tip of jaw piece 60 downward and the proximal end of jaw piece 60 upward. Accordingly, and by virtue of this parallel jaw configuration, pressure being applied to the tissue from the jaws is distributed with substantial evenness across the length of contact between the jaws and the tissue, and radiofrequency energy, when delivered, is also distributed with substantial longitudinal evenness or uniformity.

FIG. 7E shows the jaw embodiments in a partially closed position, with the jaws, as they would be when closing around a portion of relatively thin target tissue, the jaws in a parallel alignment, spaced apart by a narrow gap, reflecting the presence of thin tissue therebetween. This relative positioning of the jaws comes about at least for two reasons, as similarly described above in the context of FIG. 7D. First, the jaws are nearly but not completely closed at the level of the rotational assembly connecting the proximal aspects of the jaws. Second, this position has been arrived at by a counter clockwise pivoting of the pivotable piece 60 of lower jaw 40 around pivotable connection 75 through, or nearly through its range of angular rotation. This clockwise rotation has moved the distal tip of jaw piece 60 slightly downward and the proximal end of jaw piece 60 slightly upward. As seen in FIGS. 7A and 7B, there is a relatively small linear profile of electrode 62 visible over the base provided by proximal jaw piece 50.

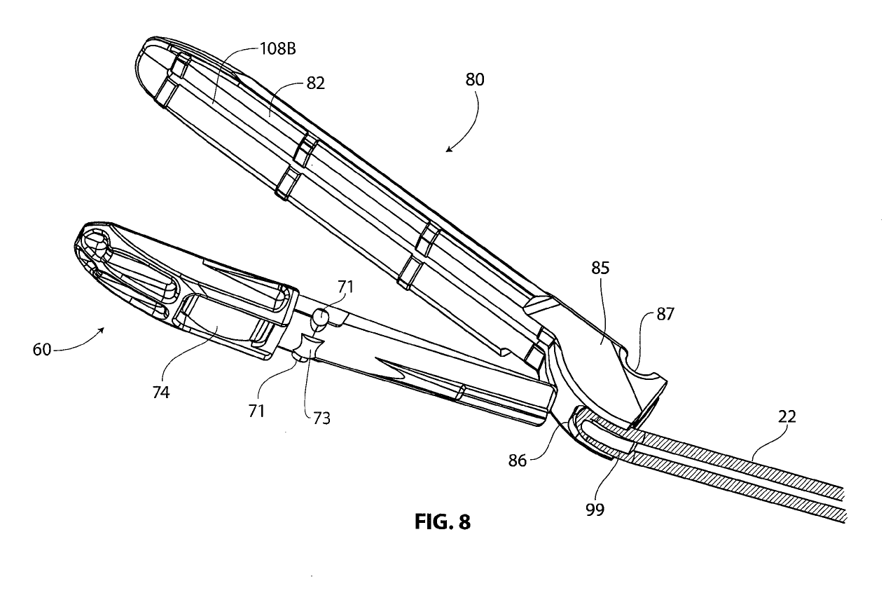

FIG. 8 is a perspective and upward looking view of a set of jaws of an embodiment of a laparoscopic electrosurgical device in an open position. More specifically, it shows an isolated upper jaw 80 and an isolated distal pivotable jaw piece 60 of a lower jaw, and an actuator wire or cable 22 looped around an attachment point 99 at the proximal end of the upper jaw. An advantage provided by this arrangement relates to ease of manufacture and assembly of this aspect of the device in that a fixed soldering point is not needed. A further structural advantage is that tension within the actuator wire is distributed through a portion of the length of the loop, rather than being focused at an attachment point. It can be seen that a distal push by actuator wire 22 would cause an upward pivoting of upper jaw 80 toward an open jaw position, and a proximal pull would cause a downward pivoting of upper jaw 80 toward a closed jaw position. At its proximal end, actuator wire 22 is connected to jaw actuator grip 15, shown in FIG. 1.

FIGS. 9A-9D provide various views of a lower jaw 40 of an embodiment of a laparoscopic electrosurgical device that includes proximal or base jaw piece 50 that is fixed with respect to the shaft and distal pivotable jaw piece 60 that is pivotably connected to the base piece. The focus of FIGS. 9A-9D relates to embodiments of a pivotable connection or assembly 75 that connects jaw pieces 50 and 60. The pivotable proximal jaw piece and the distal jaw piece are pivotably connected at pivotable joint located at a substantially central site on the pivotable piece and at a distal aspect of the proximal jaw piece.

FIG. 9A is a side view of an isolated lower jaw 40 of a laparoscopic electrosurgical device, the lower jaw including a proximal jaw piece 50, fixed with respect to the shaft, and distal pivotable jaw piece 60 mounted at a substantially central point on a distal aspect of the proximal jaw piece. It can be seen that pivotable assembly 75 includes a boss 71 of pivotable jaw piece 60 rotatably disposed in a recess 48 of base jaw piece 50. This is a bilateral arrangement, bosses 71 projecting outward on both sides of pivotable jaw piece 60, and mating recesses 48 on both sides of base jaw piece 50. This arrangement thus represents a pivotable mechanism that does not include a through pin. This arrangement further provides advantage in ease of assembly, in that the component parts can be snap fitted together.

FIG. 9B is a perspective view of an embodiment of an isolated lower jaw 40 of a laparoscopic electrosurgical device that shows a lower jaw 40 having a proximal jaw piece 50 and distal pivotable jaw piece 60 in an exploded relationship. Distal piece 60 is shown moved up and moved distally with respect to its assembled position within proximal piece 50. A boss 71 is visible on one side of pivotable jaw piece 60, and both of receptacles or recesses 48 of lower base jaw piece 50 are visible. The proximal aspect of base jaw piece 50 is sufficiently flexible that it can expand to accommodate entry of pivotable jaw piece 60. After engagement of both bosses 71 into their respective receptacles 48, the expanded base piece snaps back to its native configuration, thus securing the pivotable jaw piece in place. Also visible in this view is pivot ridge 30, centrally disposed beneath bosses 71. When assembled, pivot ridge is in contact with an upper surface of base jaw piece 50, and provides the elevation that allows pivoting to occur. FIG. 9C provides a bottom view of a lower jaw 40 of a laparoscopic electrosurgical device, showing a view of the connection between a proximal jaw piece 50 and distal pivotable jaw piece 60 assembled together. Bosses 71 of pivotable jaw piece 60 are visible within recesses 48 of lower base jaw piece 50.

FIG. 9D is an upward looking perspective view of an isolated distal pivotable piece 60 of a lower jaw 40 of a laparoscopic electrosurgical device. Bosses 71 are visible; as is pivot ridge 73. Also visible is a biasing member such as leaf spring 74 that is positioned in a recess of the lower aspect of pivotable jaw piece 60 of lower jaw piece 50. Embodiments of a biasing member disposed in this position serve to maintain a bias or default position of pivotable piece 60 such that its distal tip is pushed away from the distal end of companion fixed jaw piece 50 of lower jaw 40, and toward the distal tip of upper jaw 80, as seen, for example, in FIG. 7B. The proximal end 65 of pivotable piece 60 includes a centrally disposed longitudinal cleft, which is a part of and contiguous with blade track 108A in the lower law, as seen from a top view perspective in FIGS. 2A and 12C.

FIGS. 10A and 10B provide semitransparent side views of a lower jaw 40 of an embodiment of a laparoscopic electrosurgical device, showing a proximal base jaw piece 50 and pivotably connected to distal pivotable jaw piece 60. FIG. 10A shows the distal pivotable jaw piece 60 in its default biased position, the distal end of the distal pivotable jaw piece being pivoted to its upper end point, toward the upper jaw (not shown). This default position is maintained as a bias by a spring, as seen best in FIGS. 11A and 11B. This is the pivoted position of distal jaw piece when the jaws are open, and which is held as the jaws are closed until a point when the distal tips of the jaws first make mutual contact, such contact representing a default tip-first closure feature of the jaws.

In contrast, FIG. 10B shows the distal end of the distal pivotable jaw piece 60 pivoted toward its lower end point, the proximal end of the distal pivotable jaw piece being pivoted toward its upper end point, such a position would putting the lower jaw in a generally parallel relationship with the upper jaw (not shown). This is the pivoted position of distal jaw piece when the jaws when the jaws are closed, or generally the position when jaws are closed around tissue, particularly when they closed around thing tissue. A boss 71 and pivot ridge 73 on the pivotal jaw piece 60 can be seen. Boss 71 is positioned within receptacle or recess 48 of base jaw piece 50. The boss and receptacle arrangement and the pivot ridge together form a pivotable connection or assembly 75.

As summarized above, embodiments of the pivotable connection or assembly 75 provide a pivotable range of about 2 degrees to about 8 degrees; particular embodiments are configured to pivot within a range of about 6 degrees. The relationship between the pivoting of distal jaw piece 60 and the dynamics associated with opening and closing the jaws, with and without tissue being grasped between them, is described above in the context of FIGS. 7A-7E. Particularly clear in FIGS. 10A and 10B is the difference in elevation of the proximal aspect of pivotable jaw 60 and its electrode bearing and tissue engaging surface 62 above the upper edge of the proximal portion of base jaw piece 50.

FIGS. 11A and 11B provide side views of a lower jaw of a laparoscopic electrosurgical device that are similar to those shown in FIGS. 10A and 10B, but which have a greater degree of transparency through the distal and pivotable piece 60 of lower jaw 40. These figures focus on a biasing member 74 in the form of a leaf spring attached to an upper aspect of the distal piece of proximal and fixed jaw piece 50. Embodiments of the technology include other arrangements that would serve the same biasing function. For example, the biasing member may include other types of springs, and it could be attached to the pivotable piece of the jaw rather than the fixed piece. In the depicted example, FIG. 11A shows leaf spring 74 attached an upper aspect of the proximal jaw piece; the spring is in an expanded configuration, pushing against the distal pivotable jaw piece so as to maintain the distal pivotable piece in its default biased position whereby the distal end of the distal pivotable jaw piece pivoted to its upper end point. In contrast, FIG. 11B the spring collapsed or compressed configuration, the result of pressure being exerted on the distal end of the distal pivotable piece of the jaw, as would occur during closure of the jaw.

FIGS. 12A-12C provide various proximal looking views of the distal tips of the jaws of an embodiment of laparoscopic electrosurgical device. These views focus on mutually complementary longitudinal aligning features that prevent lateral slippage or misalignment when the jaws close, particularly when they close around a portion of target tissue. Complementary V-shaped surfaces are used in the depicted examples of longitudinal features that encourage the self-alignment of jaws, but those familiar with the art will recognize that other complementary surfaces will serve the same purpose, and as functional equivalents, are included as embodiments of the disclosed technology.

FIG. 12A is a proximal-looking perspective view of the distal tips of a closed set of jaws, while FIG. 12B is a facing view. Upper jaw 80 shows a V-shaped recession on distal tip 96; distal piece 60 of lower jaw 40 shows a V-shaped projection on its distal tip 66. The mutually complementary V-shaped profiles are represent a profile that extends substantially through the length of the respective electrode surfaces, i.e., electrode surface 82 of upper jaw 80 and electrode surface 62 of pivotable piece 60 of lower jaw 40, respectively. The full length of the respective electrode surfaces is best seen in FIG. 12C. Embodiments of the technology include configurations where the mutually complementary jaw surfaces do not extend the full length of the jaws, and the shape of the complementary surfaces need not necessarily be of consistent shape through the length of the jaws.

FIG. 12C is a proximal-looking perspective view of a distal aspect of an open set of jaws of laparoscopic electrosurgical device showing a V-shaped projection on the lower jaw, and a V-shaped recession on the upper jaw, as well as a central longitudinally-oriented gap in both V-shaped surfaces that form a through passage for a blade that is distally advanceable when the jaws are in a closed position. FIG. 12C further shows insulative strips 92 arranged across electrode tray or bipolar electrode surface 82 of upper jaw 80. Additionally, centrally disposed longitudinal gaps are visible in both the upper jaw and lower jaw. Gap 108A in lower jaw piece 60 and gap 108B in upper jaw 80 collectively form a through path for distal passage 106 of for blade 105 (not seen here, but shown in FIG. 2B).

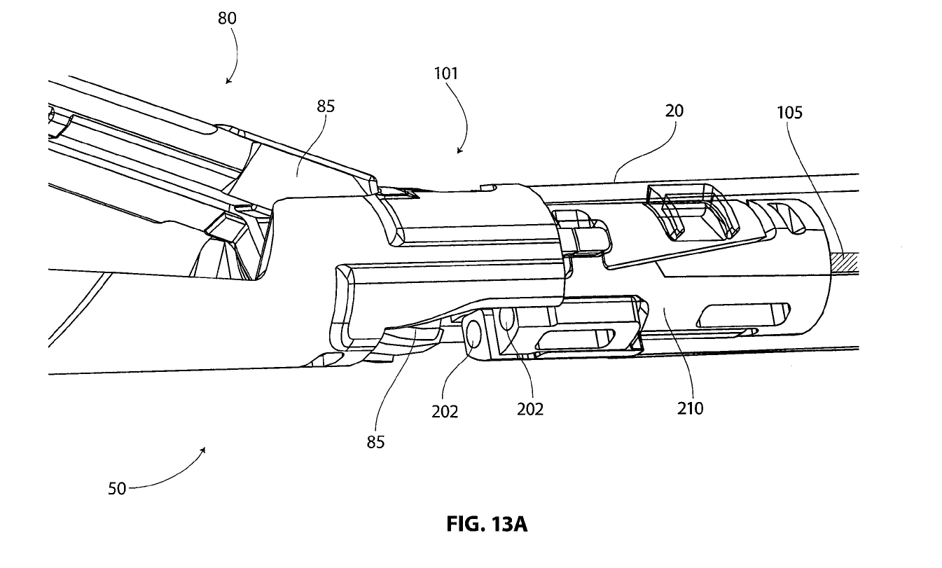

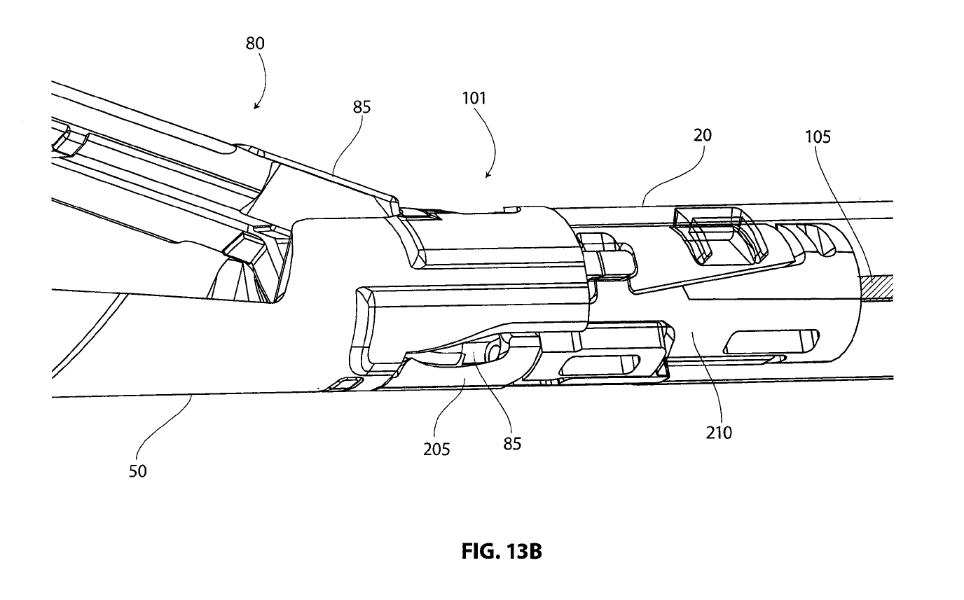

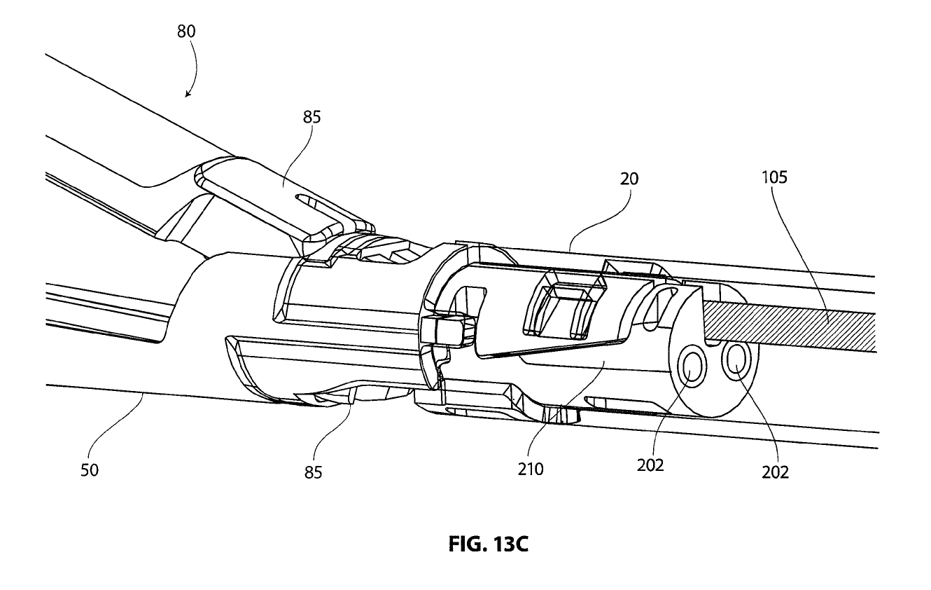

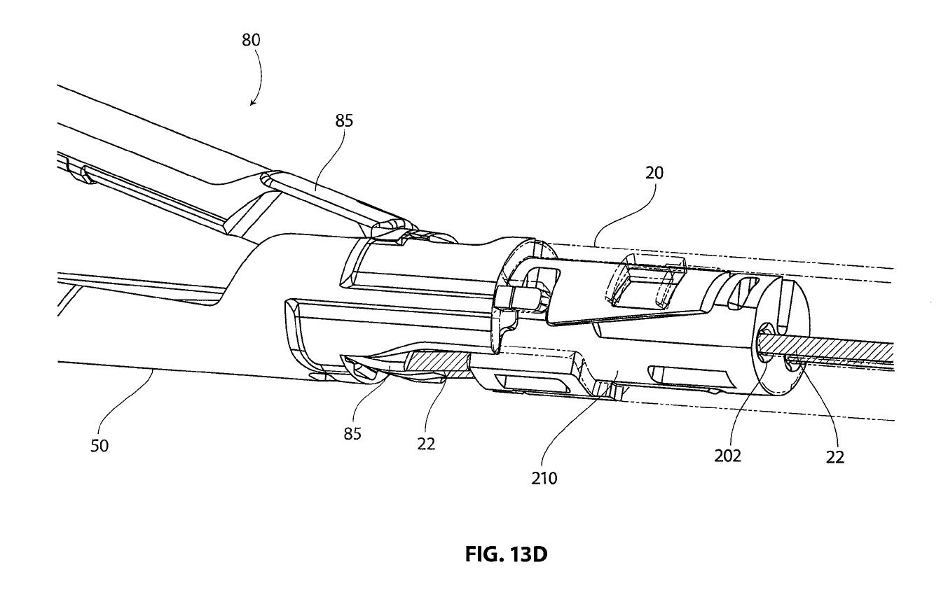

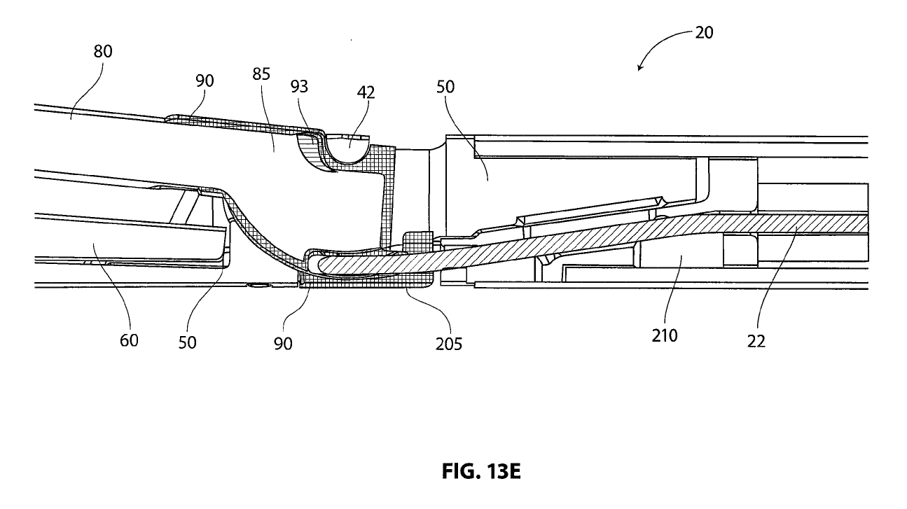

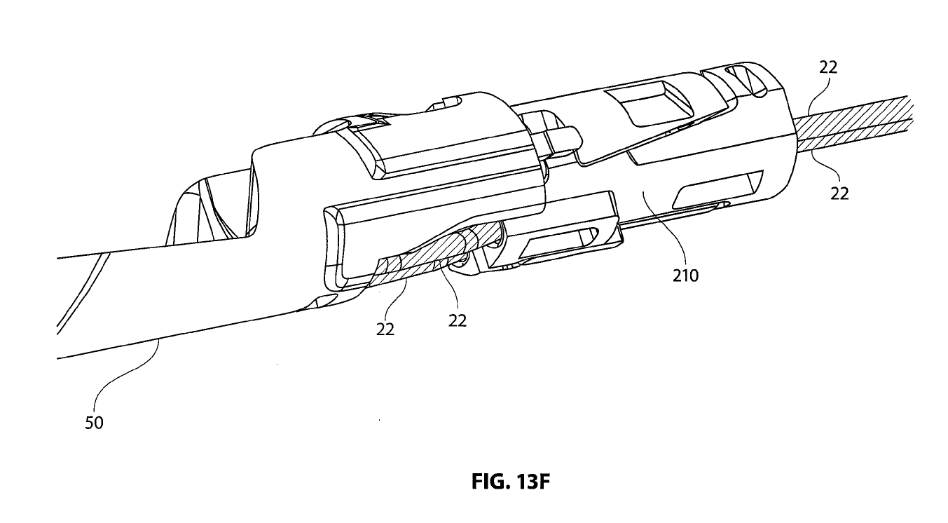

FIGS. 13A-15C all relate to in various ways to aspects of the junction between the proximal end of a jaw set and the distal end of a shaft, and to the separate and insulated electrical pathways to the upper jaw and lower jaw, respectively, per embodiments of the technology. FIGS. 13A-13F provide various views of an embodiment of an electrosurgical device that show aspects of the proximal portion of a set of jaws and the very distal portion of the shaft through which jaw actuator cables or wires transit. FIG. 13A provides an exposed proximal looking perspective view of a wire isolator or channelizing unit 210 disposed at the bottom (in this view) of the distal end of shaft 20. This isolator unit 210 guides the twinned actuator wires (not shown) from the center of the shaft to this cross-sectionally eccentric position such that the wire is positioned for its attachment to a proximal site of the arcuate track 85 of upper jaw 80 (see FIG. 8). Twin wire channels 202 may be seen in the distal face of channelizing unit 210. As noted above, embodiments of the actuator wire for upper jaw 80 also convey electrical current to upper jaw 80. Another function of wire isolator unit 210 is thus to insulate shaft 20 and proximal base 50 of the lower jaw from current being conveyed to the upper jaw.

FIG. 13B has the same perspective orientation as that of FIG. 13A, but shows a cable retaining plate 205 in place over an area where cables emerge from a central transit through the shaft and are diverted to an eccentric site, where they are attached to a proximal aspect of the pivotable upper jaw. Cable retaining plate 205 secures cables through this portion of their path, and also provides electrically insulates the wires within this space. FIG. 13C is a distal looking transparent view that shows a cable isolator unit with parallel cable channels. FIGS. 13C and 13D both provide a view of blade 105 and its path through isolator unit 210, as well as the distal openings of wire channels 202. FIG. 13D provides a view similar to that of FIG. 13C, but with the cables 22 in place.

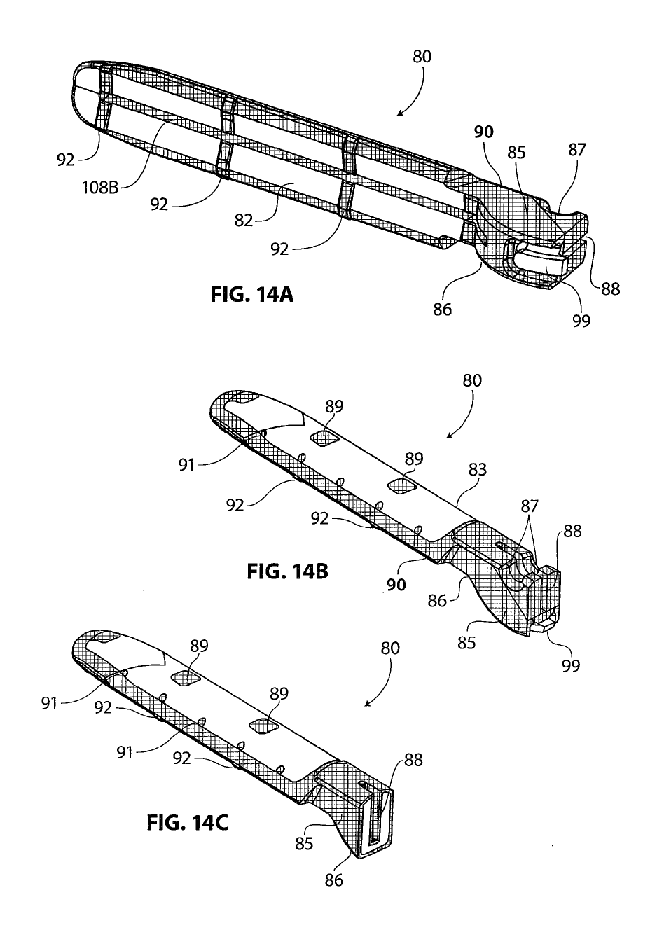

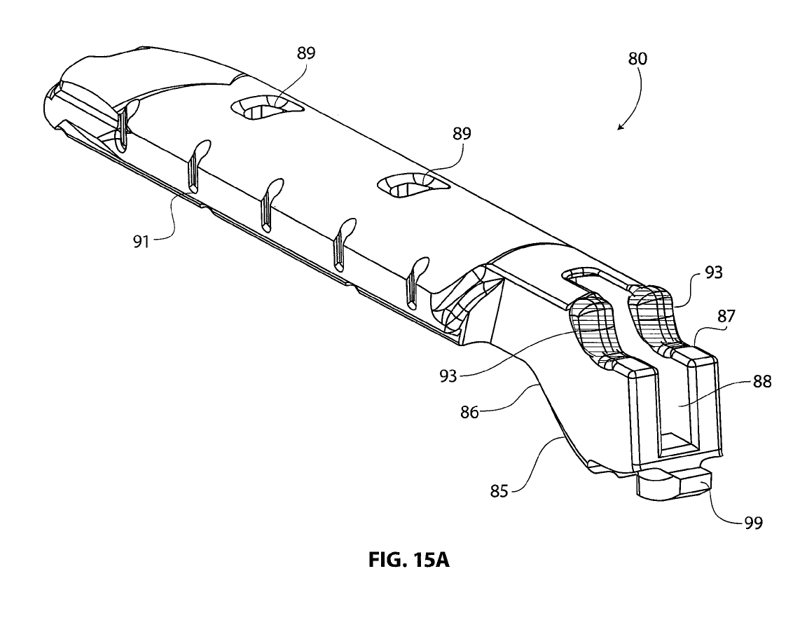

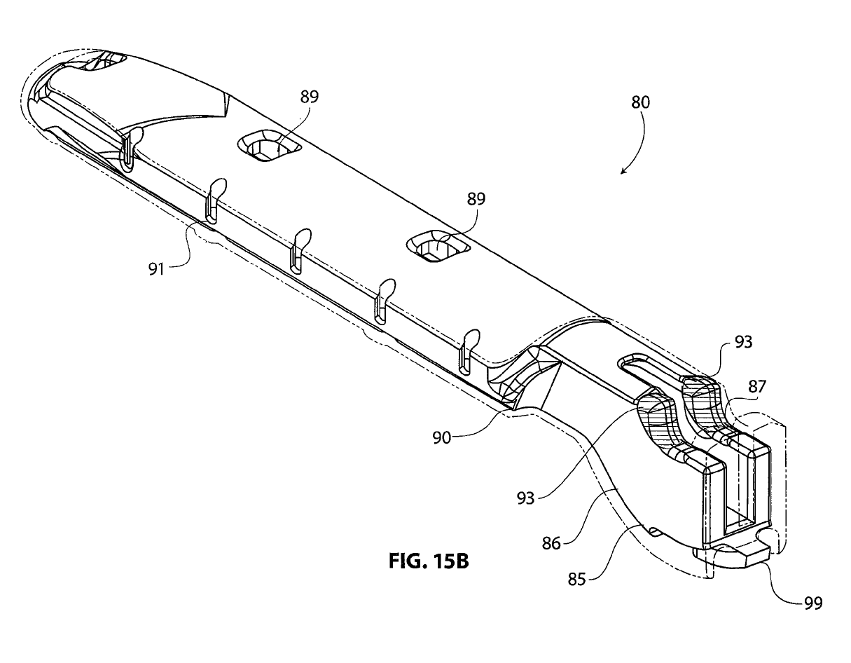

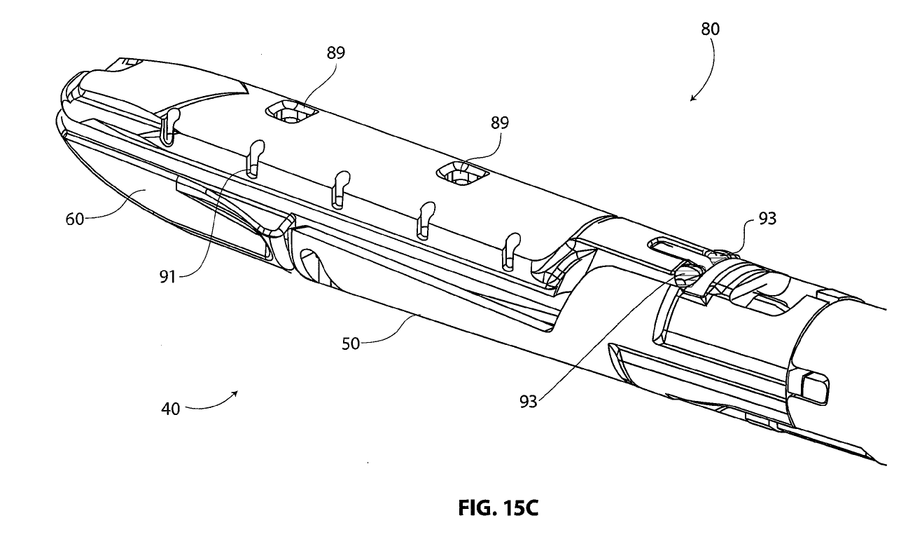

FIG. 13E is a longitudinal section side view, slightly offset from midline, showing the paths of cables 22 through the distal portion of the shaft and into the proximal aspect of the jaws. The closer of the twinned cables 22 can be seen being channeled from its substantially central position within the main body of the shaft to a peripheral position at the very distal end of the shaft. As cable 22 transitions into the proximal base of the jaws, it wraps around attachment site 99 of the base of upper jaw 80. Polymer layer 90 can be seen as an outline surrounding a major portion of the arcuate track portion 85 of upper jaw 80, however cable attachment site is not covered with polymer. The bare aspect of cable attachment site 99 can also be seen in FIGS. 14A, 14B, and 15A, and 15B. Other aspects of the arcuate track portion of the upper jaw that engage surfaces of the base portion 50 of the lower jaw are coated with polymer 90 such that upper and lower jaw surfaces are insulated from each other. Accordingly, twinned cable 22 makes direct electrical contact with upper jaw 80 to the exclusion of contact with lower jaw piece 50. Cable retaining plate 205 (see FIG. 13B) is formed from plastic, and it thus also serves an insulative function.

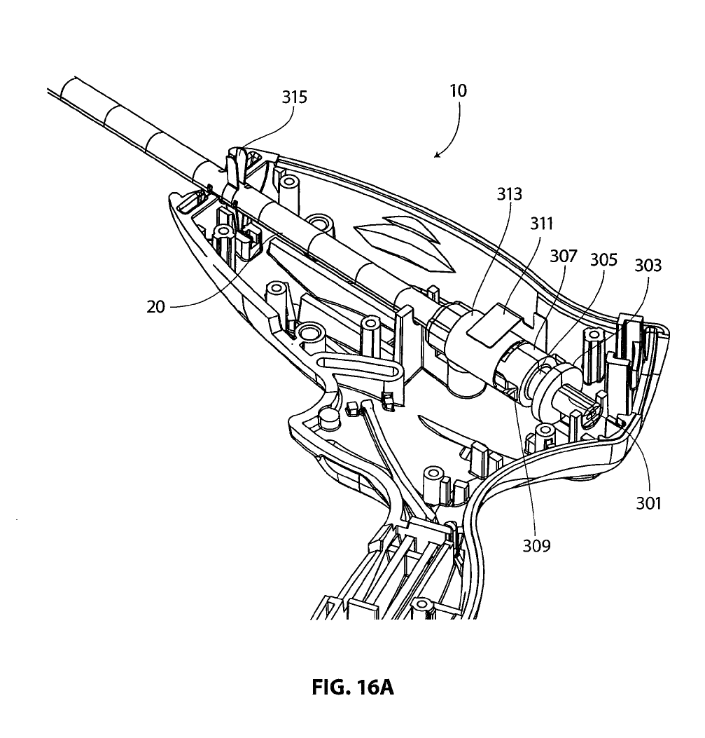

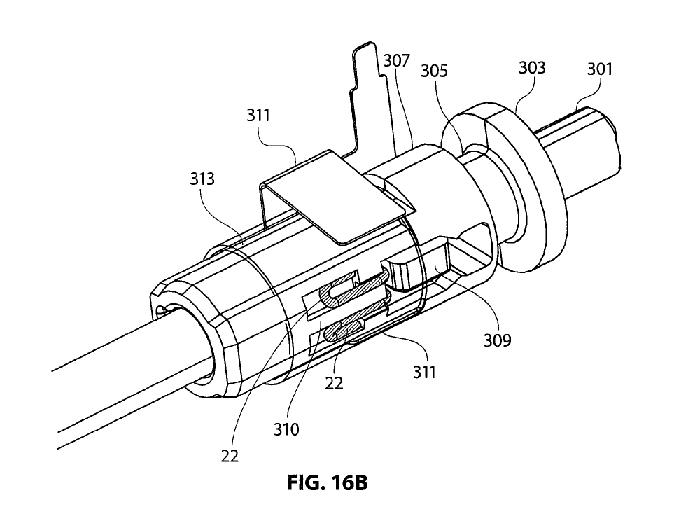

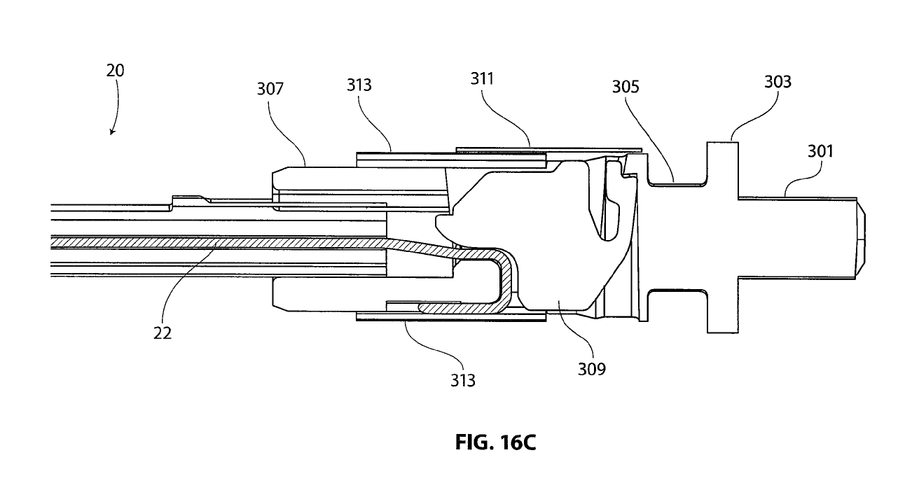

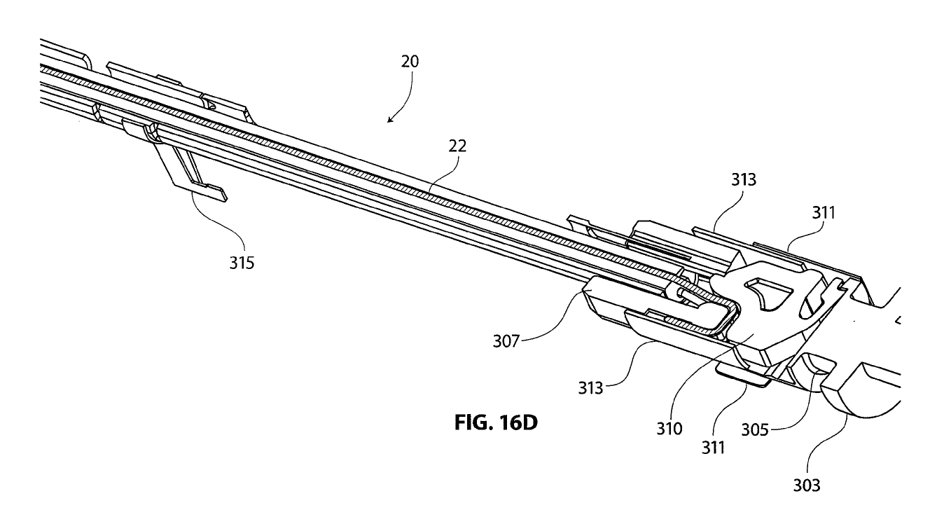

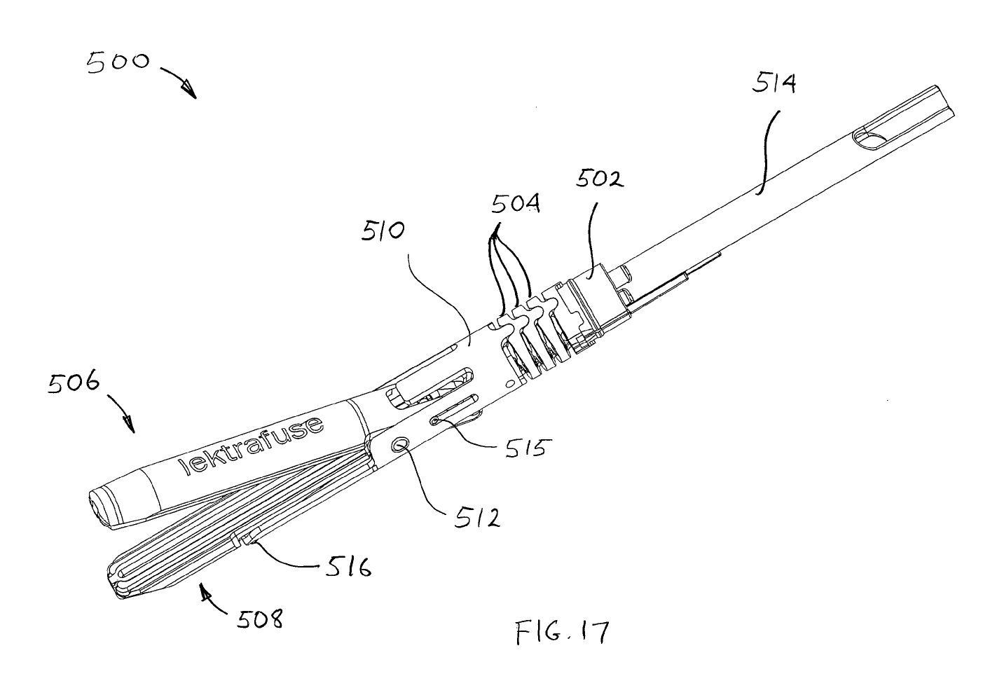

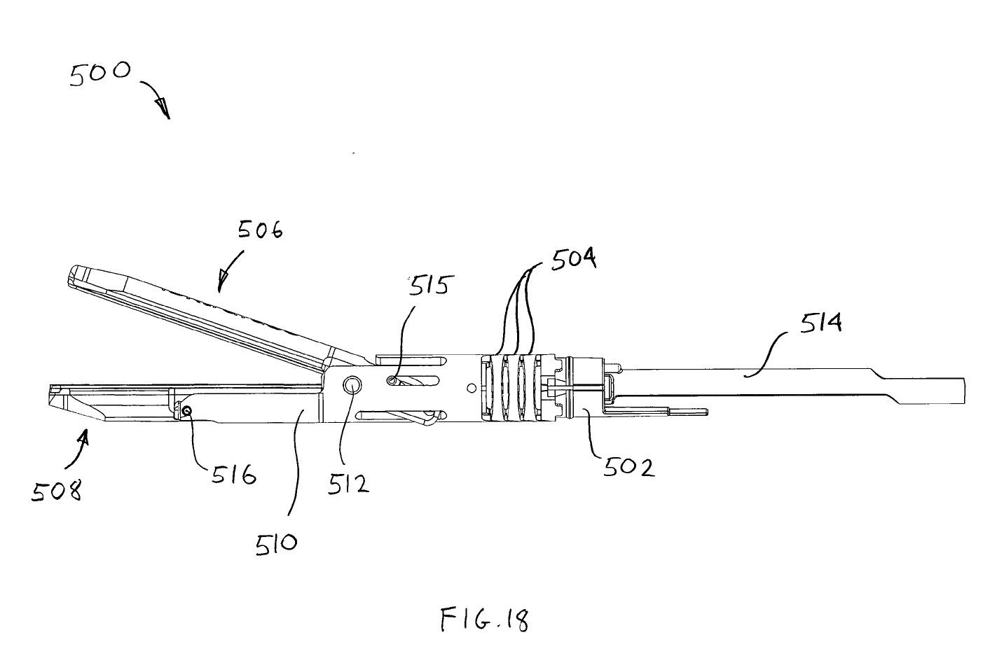

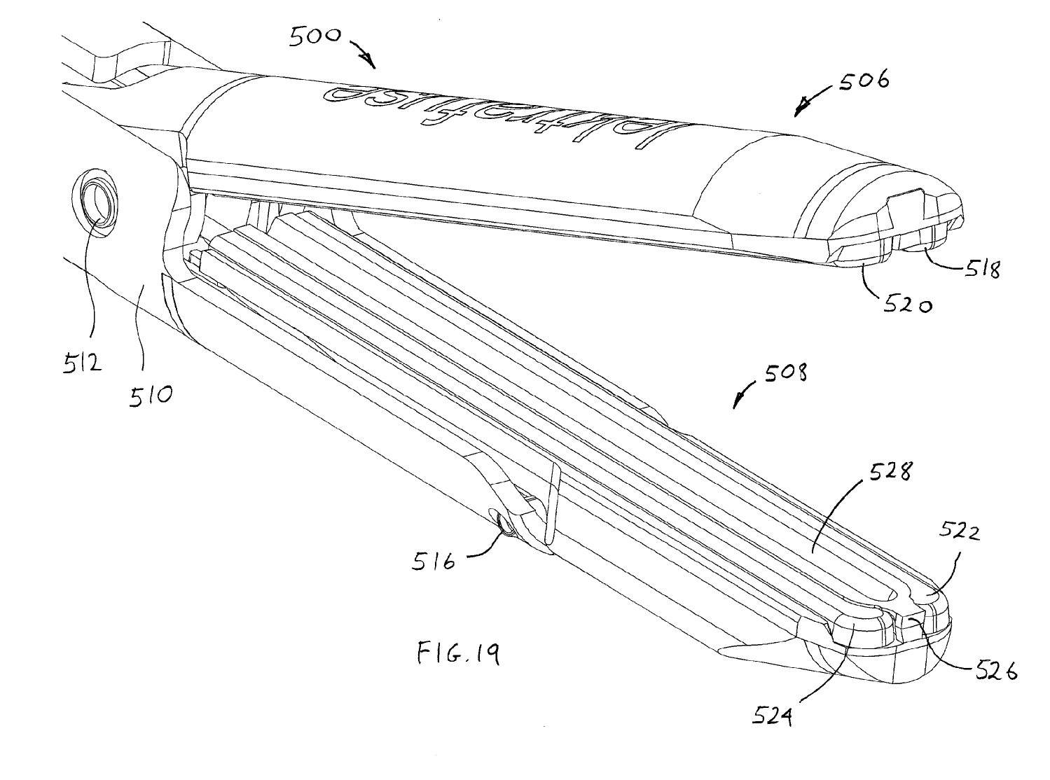

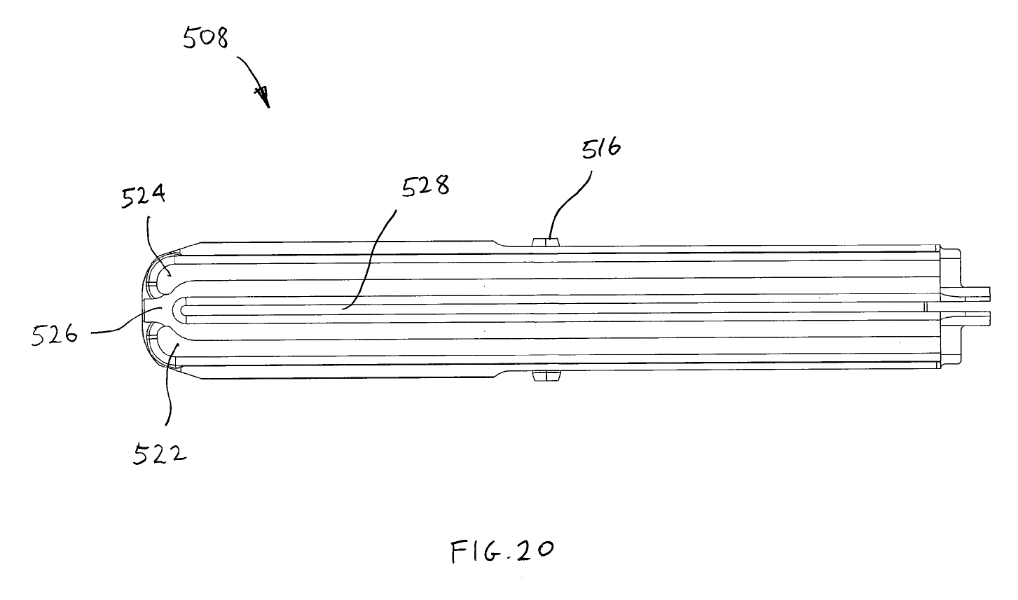

FIG. 13F is proximal looking perspective view of the proximal end of a lower jaw piece 50 that is inserted into the distal end of a shaft, further showing engagement of the proximal end of the shaft with a cable isolator unit. FIG. 13E and FIG. 13F also generally depict a distal aspect of the electrical path that provides radiofrequency energy to the upper jaw, to the exclusion of the lower jaw. The electrical path that provides radiofrequency to the lower jaw is the shaft 20 as a whole. Aspects of the proximal portions of the electrical paths to the upper and lower jaws are shown in FIGS. 16A-16D.

FIGS. 14A-14C provide various non-transparent views of an embodiment of an insulative layer 91 that covers aspects of an upper jaw 80 of an electrosurgical device. FIG. 14A is a bottom perspective view of an embodiment of an upper jaw of that shows plastic insulator layer overlaying aspects of an electrode. FIG. 14B is a top perspective view of an embodiment of an upper jaw of an electrosurgical device that shows polymer insulator layer overlaying peripheral and proximal aspects of the electrode. FIG. 14C is a top perspective view of an embodiment of an upper jaw that shows polymer insulator layer overlaying the electrode, with the proximal portion of an jaw truncated to expose a cross section. FIGS. 14A-14C show polymer layer 90 (bolded indicator) in a relatively light rendering that covers a major portion of upper jaw 80; uncoated metal is shown in a darker rendering. These figures also provide a good view of aspects of the arcuate track 85 portion of upper jaw 80, including the upper and smaller arcuate track surface 87, the lower and greater arcuate track surface 86, and a central slot 88, which is contiguous with blade track 106 (as also seen in FIG. 12C).

In FIG. 14A, polymer coating 90 is seen around the periphery of the exposed metal electrode surface 82 and actuator attachment site 99 in FIG. 14A. The more lightly rendered polymer overlay also takes the form of insulative strips 92 that are arranged across the surface of electrode 82. The thickness of the polymer coating 90 is in the range of about 0.005 inch to about 0.015 inch. The polymer layer that takes the form of insulative strips 92 stands off from the broader electrode surface 82 by about 0.004 inch to about 0.008 inch, but its overall thickness is greater because it is positioned in a trough, as seen in FIG. 5A (trough 84 within electrode surface 142).

FIGS. 14B and 14C show exposed or uncoated metal on the upper surface 83 of upper jaw 80. FIG. 14B shows that insulative layer 90 fully coats the proximal aspect of upper jaw 80, including the surfaces of arcuate track portion 85. Receptacles 89 on the upper aspect of the jaw are filled with polymer 90, as the polymer fills these receptacles such that it is a continuous fill from the lower electrode side of the jaw (as seen in FIG. 14A) through to a top surface exposure.

FIG. 14C differs from FIG. 14B in that the proximal aspect of the jaw is truncated with a cross section exposure 85C just distal of smaller or upper concentric surface of arcuate track 85. FIGS. 14B and 14C also show insulator strip anchoring receptacles 89 on the top of jaw 80. These receptacles penetrate the metal and fill with polymer during the coating process, anchoring the coating against the electrode surface. On the bottom surface of the electrode, receptacles 89 are positioned within blade track 108B (see FIG. 14A). Peripheral anchoring recesses 91 are arranged around the edge of jaw 80, also serving to stabilize polymer layer 90 in place.

FIGS. 15A-15C provide various views of an embodiment of an insulative layer 90 that covers aspects of an upper jaw of an electrosurgical device and which includes areas of ceramic reinforcement 93 at particular sites that can be subject to abrasive stress or erosion. These abrasively stressed sites are on the upper surface of arcuate track 85 (more particularly the smaller concentric surface 86) of upper jaw 80. When the jaws pivot, these sites rotate against the upper concentric surface of the arcuate track of the lower jaw (see FIGS. 3A-3C and FIG. 8). The stress applied to this area of rotational engagement of the upper and lower jaws comes from the tension that can be applied by the jaw actuator wire.

FIG. 15A is a top perspective view of an embodiment of an upper jaw that shows ceramic points 93 overlaying the electrode at abrasive stress points. This view does not include an overlaying polymer layer. FIG. 15B is a top perspective view of an embodiment of an upper jaw that shows points of ceramic 93 overlaying the electrode at abrasive stress points as they are embedded in a more extensive polymer layer 90. FIG. 15C is a top perspective view of an embodiment of a pair of closed jaws that shows ceramic points 93 overlaying the electrode at abrasive stress points as they are embedded or disposed within a more extensive polymer layer 90.

FIGS. 16A-16D show various views of the proximal portion of an embodiment of a rotatable shaft 20 and electrical and mechanical components associated with the shaft that are housed in the handle 10 of an electrosurgical device. FIG. 16A is an exposed distal looking perspective view of a handle of an embodiment that shows aspects of the proximal end of a rotatable shaft. FIG. 16B is a proximal looking perspective view of an isolated proximal end of a rotatable shaft. FIG. 16C is a midline sectional side view of an isolated proximal end of a rotatable shaft. FIG. 16D is a midline exposed sectional view of a portion of the rotatable shaft that is housed in the handle.

As seen in these various views, the proximal end of shaft 20 terminates into a proximal shaft-associated assembly that includes an actuation collar 307 around which is slidably wrapped within a power tube 313. Proximal to actuation collar 307 are a control flange 303 and a control post 301. A jaw actuator engagement groove 305 is disposed between control flange 303 and control post 301. The actuation collar and its wrap around power tube are disposed within the partially enclosing U-shaped proximal electrical connector 311. The actuation collar and power tube are both rotatable and slidable within the proximal electrical connector. Actuation of the rotation of the shaft (and the actuation collar and power tube) is controlled by rotation actuator 12, as shown in FIGS. 1A-1D, but not shown in this view. Actuation of the distal-proximal slidability of the collar and power tube is controlled by a mechanical linkage that is ultimately connected to jaw actuator grip 15 as shown in FIGS. 1B-1D. The jaw actuator linkage engages the shaft-associated assembly within groove 305.

The proximal electrical connector 311 delivers radiofrequency electrical energy to power tube 313 through a secure but slidable contact that is maintained regardless of the rotational position of the power tube, and regardless of the distal to proximal translational position of the power tube. Electrical energy is conveyed by this path from a generator that is part of a larger electrosurgical system to cables 22 that terminate proximally within actuation collar 307 at a proximal cable attachment site 310. A collar plug 309 that fills an asymmetric space within a proximal aspect of actuation collar 307 serves in several mechanical capacities, one of them being to secure cables 22 in their attachment to attachment site 310. Cables 22 terminate distally in an attachment to an upper jaw, as shown in FIG. 8.

Electrical energy is also conveyed to distal electrical connector 315 from a system generator, and electrical connector 315 delivers energy to the shaft 20, which then conducts energy to the lower jaw piece 50. By these approaches, electrical paths to the upper jaw and lower jaw, respectively are segregated within the handle. Separate paths are maintained throughout the main body of the shaft, where electrical energy to the upper jaw travels through the centrally disposed twin cables 22, and where electrical energy to the lower jaw travels through the columnar shaft 20. Segregation of these two paths at the junction of the shaft and the jaws is described above in the context of FIGS. 13A-13F.

Referring to FIGS. 17-23, another embodiment of jaw construction is shown. As best seen in FIGS. 17 and 18, jaw assembly structure 500 is shown pivotably coupled to a shaft bushing 502, which in turn resides on the distal end of an instrument shaft (not shown, for clarity of illustration.) Three pivotable vertebrae 504 allow jaw assembly 500 to articulate left and right relative to shaft bushing 502. Jaw assembly 500 includes an upper jaw assembly 506 and a lower jaw assembly 508. Upper jaw assembly 506 is movably attached to jaw housing 510 with pivot pin 512. Jaw actuator 514 is coupled to upper jaw assembly 506 inside jaw housing 510 with pin 515 to allow upper jaw assembly 506 to be pivoted from an open position as shown to a closed position in which it contacts lower jaw assembly 508. Lower jaw assembly 508 is pivotably attached to the distal end of jaw housing 510 by pin 516 to allow lower jaw assembly 508 to conform to tissue grasped between the upper and lower jaws and apply uniform pressure to the tissue along the length of the jaws.

Referring to FIGS. 19 and 20, the upper jaw assembly 506 includes a left electrode 518 and a right electrode 520. Lower jaw assembly 508 also includes a left electrode 522 and a right electrode 524. When the upper jaw assembly 508 is moved into a closed position adjacent to lower jaw assembly 508, the upper left electrode 518 lines up over the lower left electrode 522 and forms a first electrode pair. Similarly, the upper right electrode 520 lines up with the lower right electrode 524 to form a second electrode pair. Radio frequency energy may be applied across each of the two electrode pairs to seal tissue grasped between the upper and lower jaws. A single, unitary, U-shaped standoff member 526 is provided on the lower jaw to maintain a predetermined gap between the upper and lower electrodes, as will be subsequently described in more detail. Standoff 526 has a longitudinal slot 528 provided down through its middle to allow a knife (not shown) to be advanced through the tissue grasped between the jaws after the tissue has been sealed.

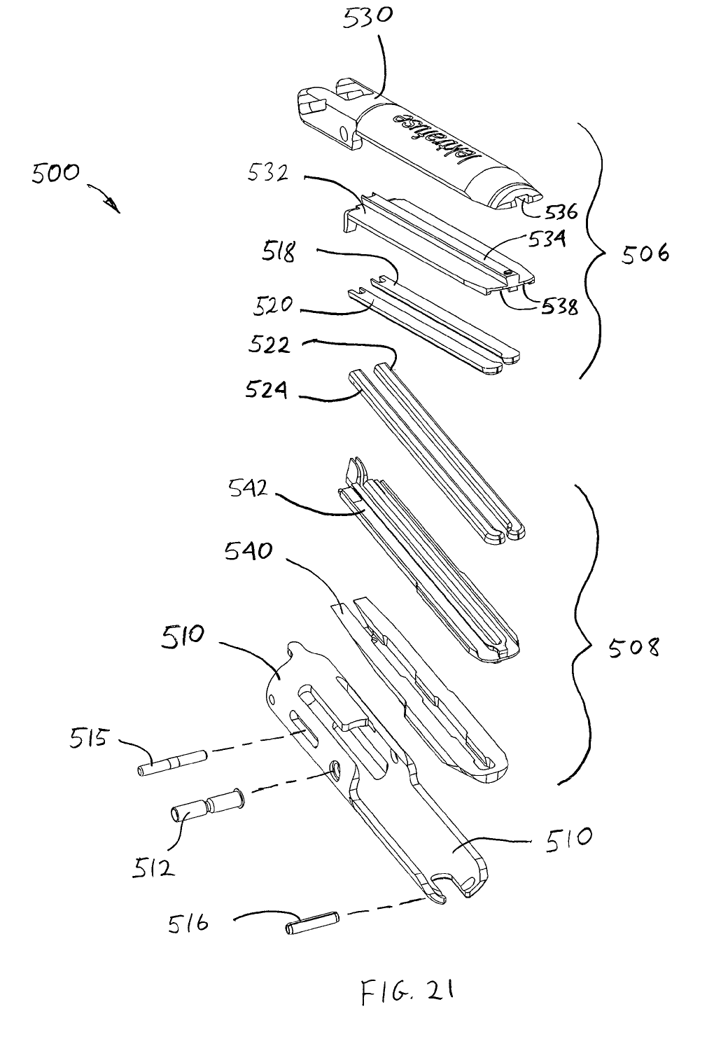

Referring to FIG. 21, an exploded view is provided showing components of jaw assembly 500. Upper jaw assembly 506 includes upper jaw arm 530, upper carrier 532, and upper electrodes 518 and 520. Upper carrier 532 is provided with a longitudinal dove-tailed ridge 534 along its upper surface. Upper jaw arm 530 is provided with a mating dove-tailed slot 536 along its lower surface for receiving ridge 534 to secure upper carrier 532 to upper jaw arm 530. Similarly, two dove-tail slots 538 are provided in the bottom surface of upper carrier 532 for securing dove-tail shaped upper electrodes 518 and 520 therein.

Lower jaw assembly 508 may be constructed in a manner similar to that of upper jaw assembly 506, and includes a lower jaw arm 540, a lower carrier 542, and lower electrodes 522 and 524, with similar dove-tail features.

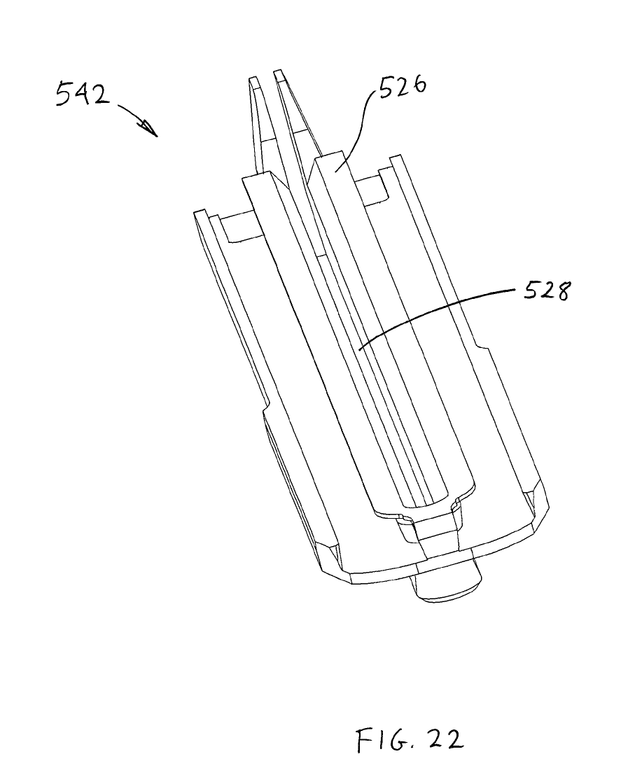

Referring to FIG. 22, an enlarged perspective view of lower carrier 542 is shown. As shown, standoff member 526 may be integrally formed with the center portion of lower carrier 542.

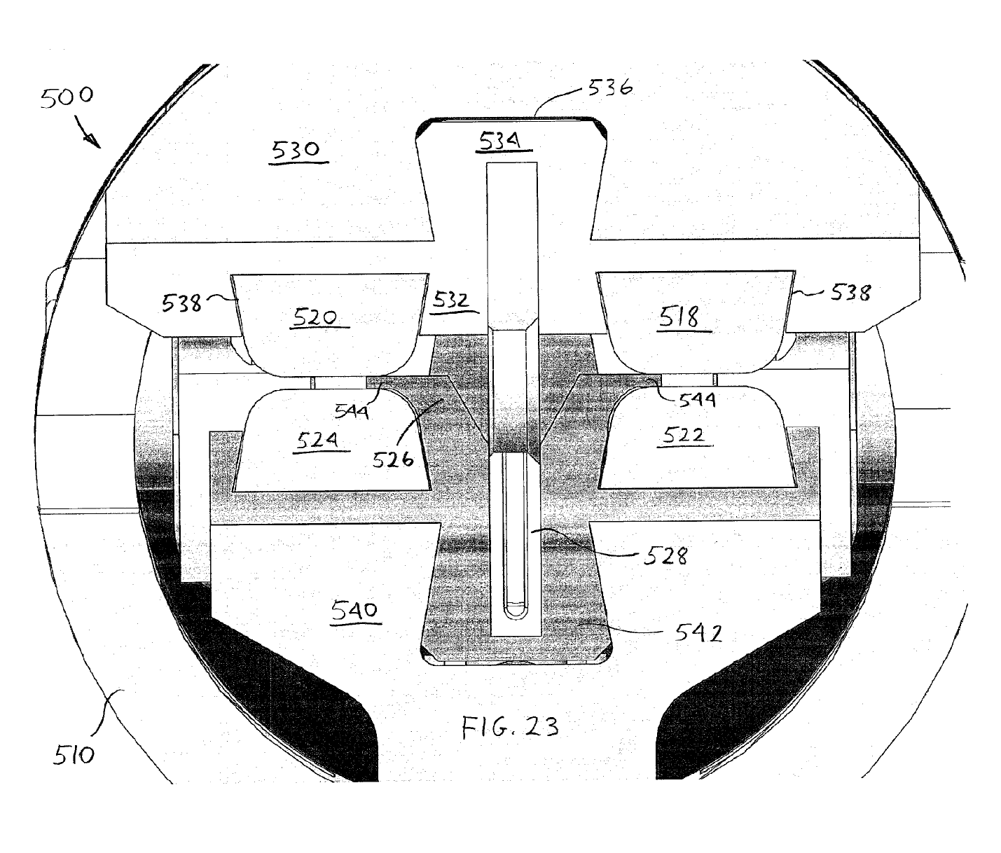

Referring to FIG. 23, a cross-sectional view taken transversely across the closed jaw assembly 500 and looking proximally down the central longitudinal axis of the shaft of the instrument. As can be seen, standoff 526 is formed integrally with lower carrier 542 and has a U-shaped region 544 that resides between upper electrodes 518, 520 and lower electrodes 522, 524. In some embodiments, region 544 of standoff 526 keeps the upper and lower electrodes a uniform distance apart of about 0.006 to 0.009 inches. In other embodiments, region 544 of standoff 526 keeps the upper and lower electrodes a uniform distance apart of about 0.005 to 0.007 inches.

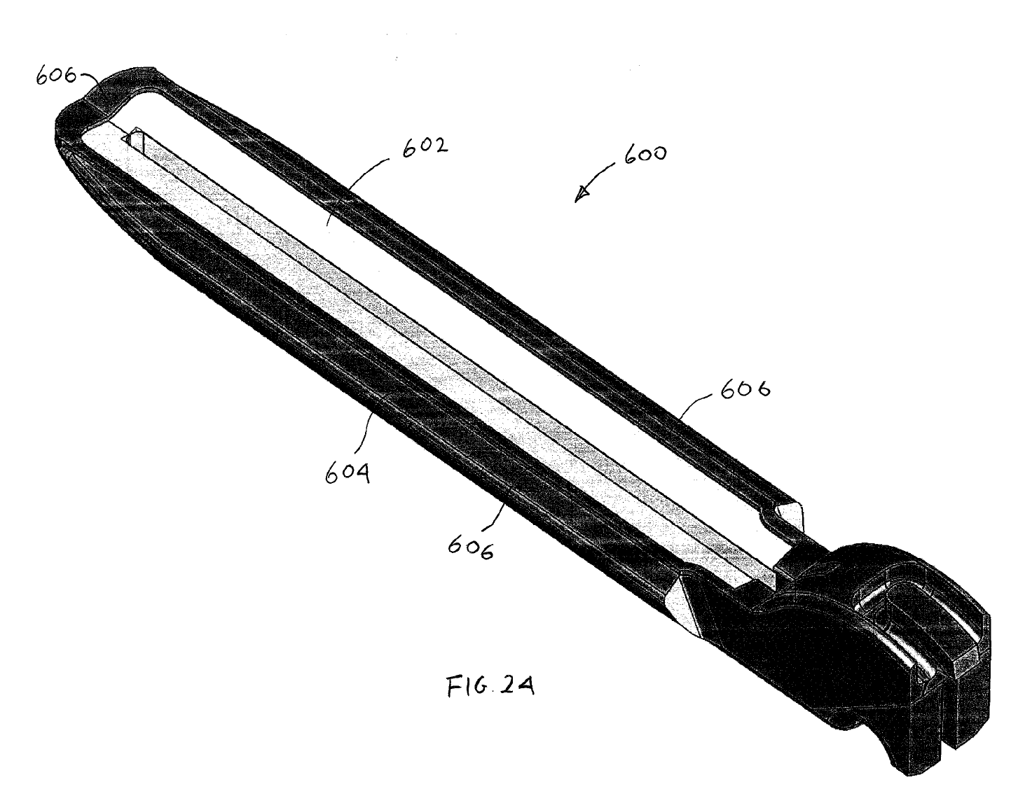

Referring to FIGS. 24-31, additional embodiments of an upper jaw with a single standoff member are shown. Referring first to FIG. 24, a first jaw configuration 600 is shown. Upper jaw assembly 600 is provided with a central conductive body 602 which is overmolded with a non-conductive portion 604. Non-conductive portion 604 includes a raised lip 606 that extends around the periphery of conductive body 602 and covers a small edge portion of the face of conductive body 602. The overmolded lip 606 is interposed between the upper electrode (i.e. conductive body 602) and the lower electrode (i.e. the lower jaw, not shown) when the upper and lower jaws come together in a closed position. Lip 606 may be held to tight tolerances so as to keep the spacing between the upper and lower electrodes within a predetermined range, as previously described.

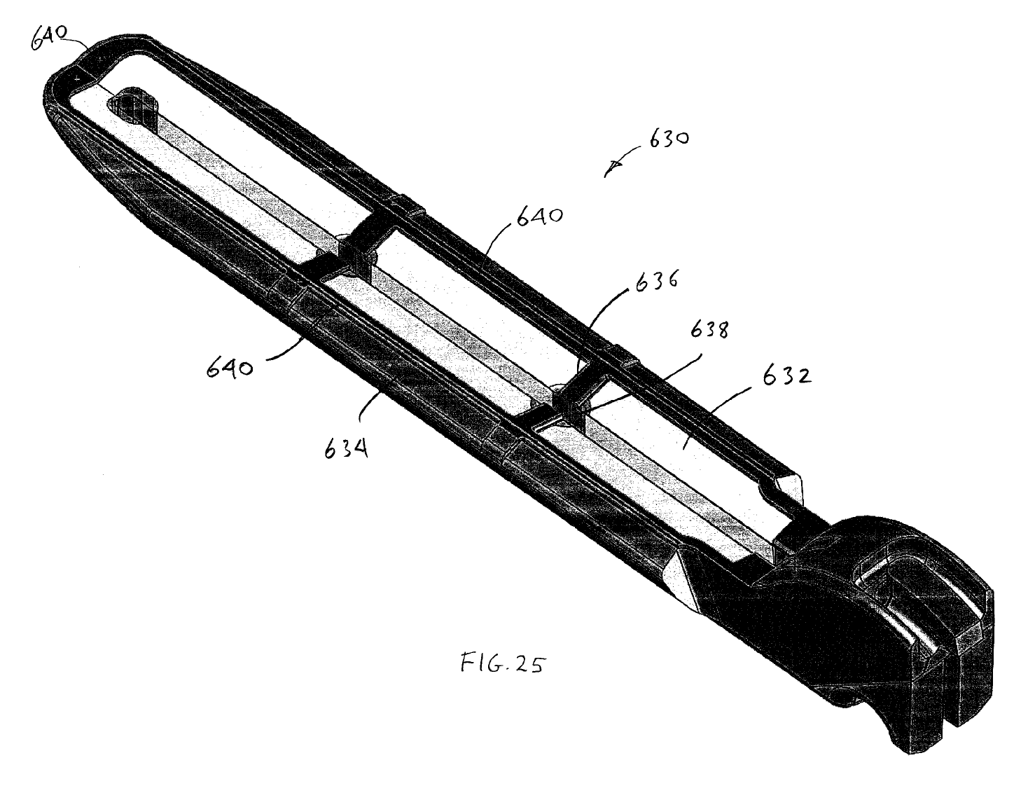

Referring to FIG. 25, a similar jaw structure 630 is shown, also having a central conductive body 632 and an overmolded non-conductive portion 634. In this embodiment, cross straps 636 are provided across portions of the electrode face and connect with overmolded plugs 638 that pass through central body 632. This arrangement helps ensure that overmolded portion 634 does not separate from central body 632 during use. Cross straps 636 may protrude a greater distance, a lesser distance, or the same distance (as shown) from the electrode surface as raised lip 640.

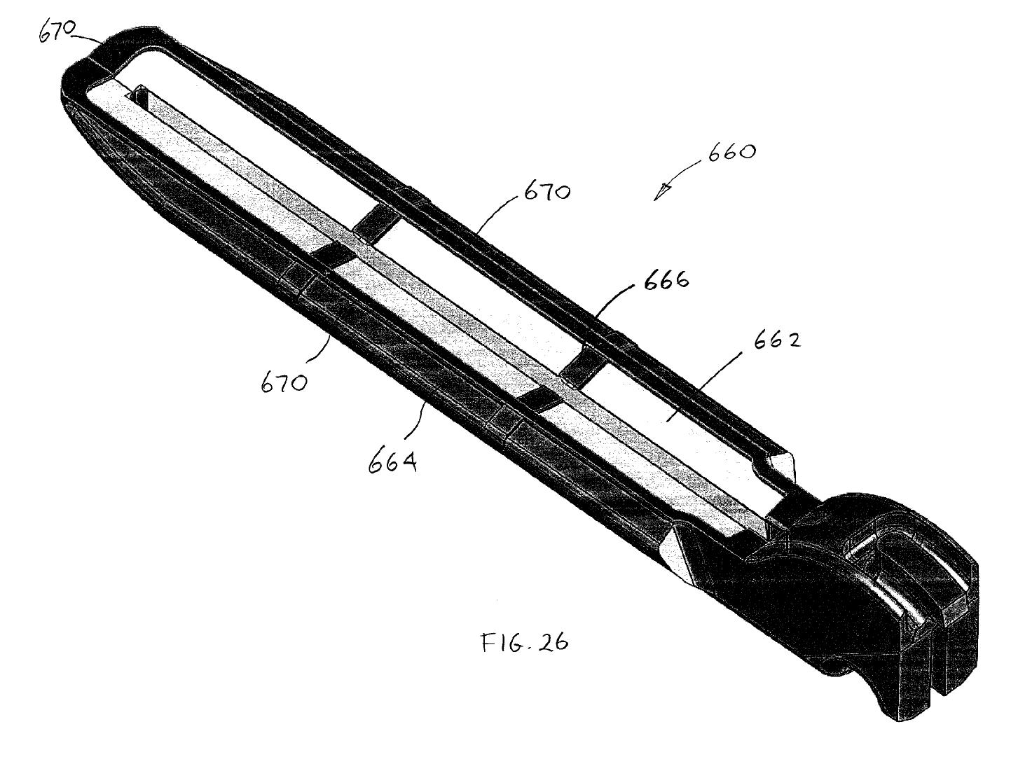

Referring to FIG. 26, a similar jaw structure 660 is shown, also having a central conductive body 662 and an overmolded non-conductive portion 664. In this embodiment, cross straps 666 are provided across portions of the electrode face but do not connect with any overmolded plugs. This arrangement also helps ensure that overmolded portion 664 does not separate from central body 662 during use. Cross straps 666 may protrude a greater distance, a lesser distance (as shown), or the same distance from the electrode surface as raised lip 670.

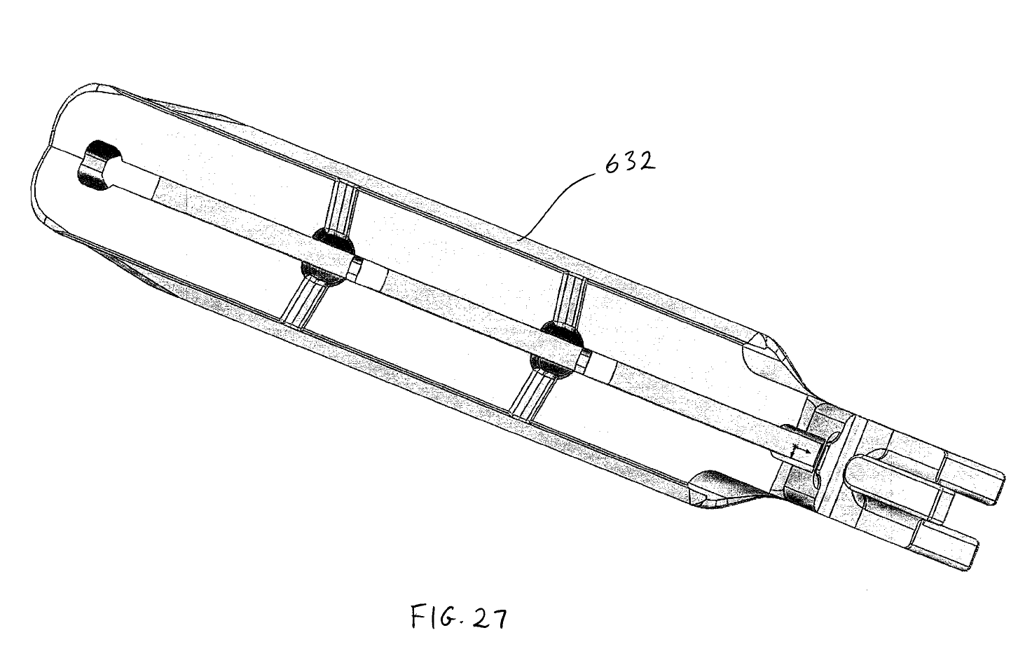

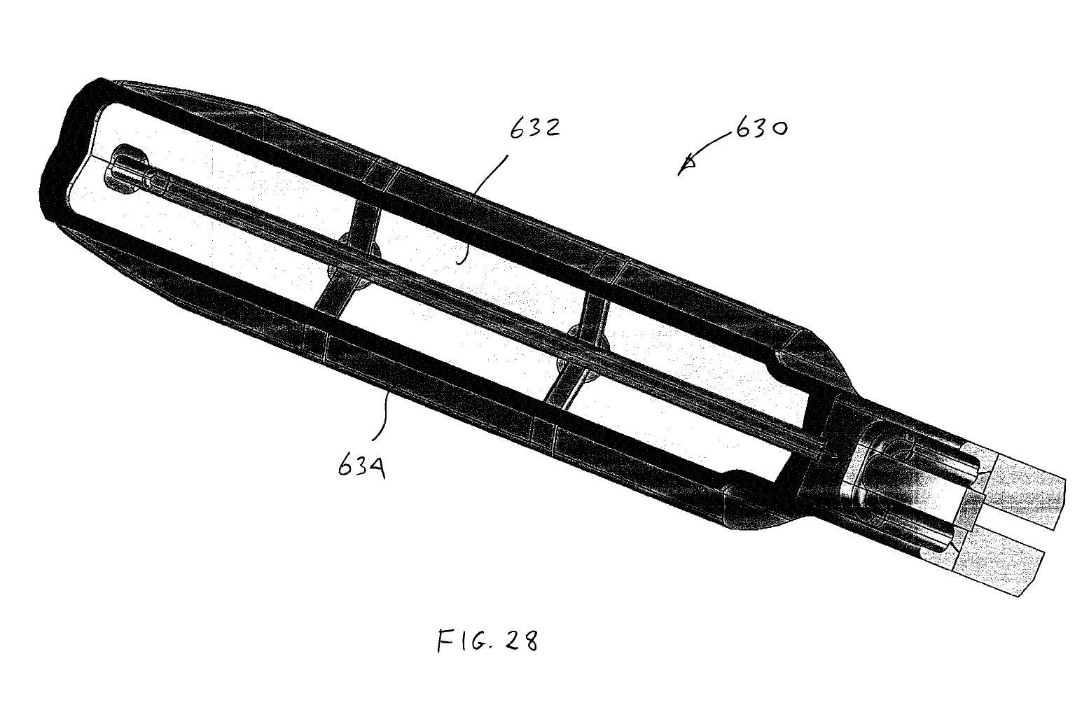

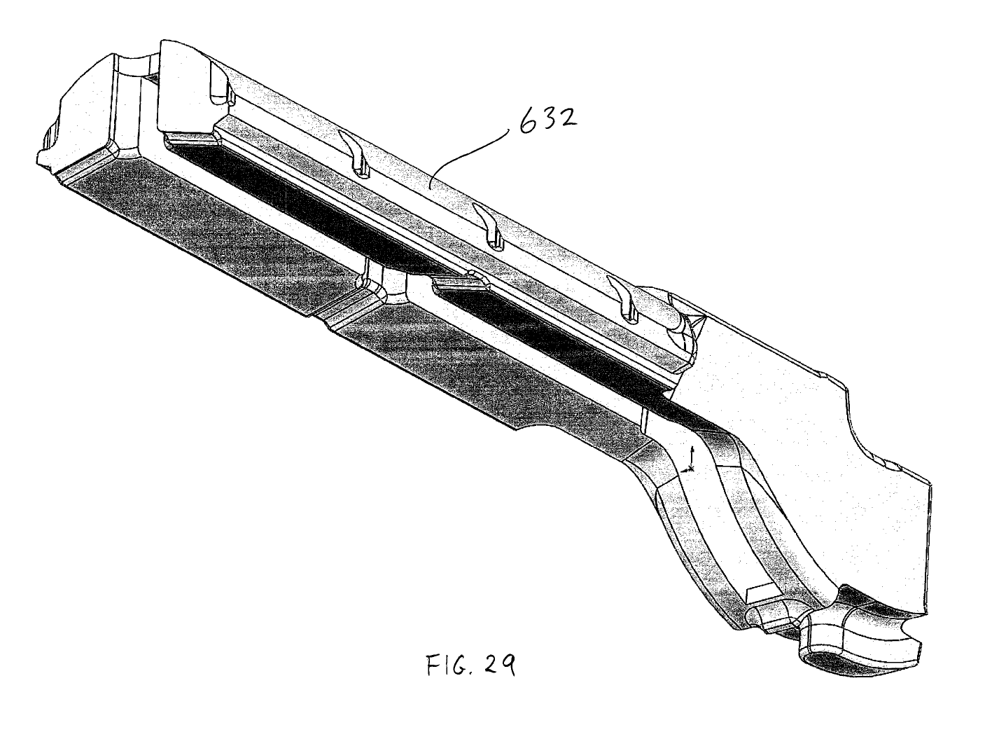

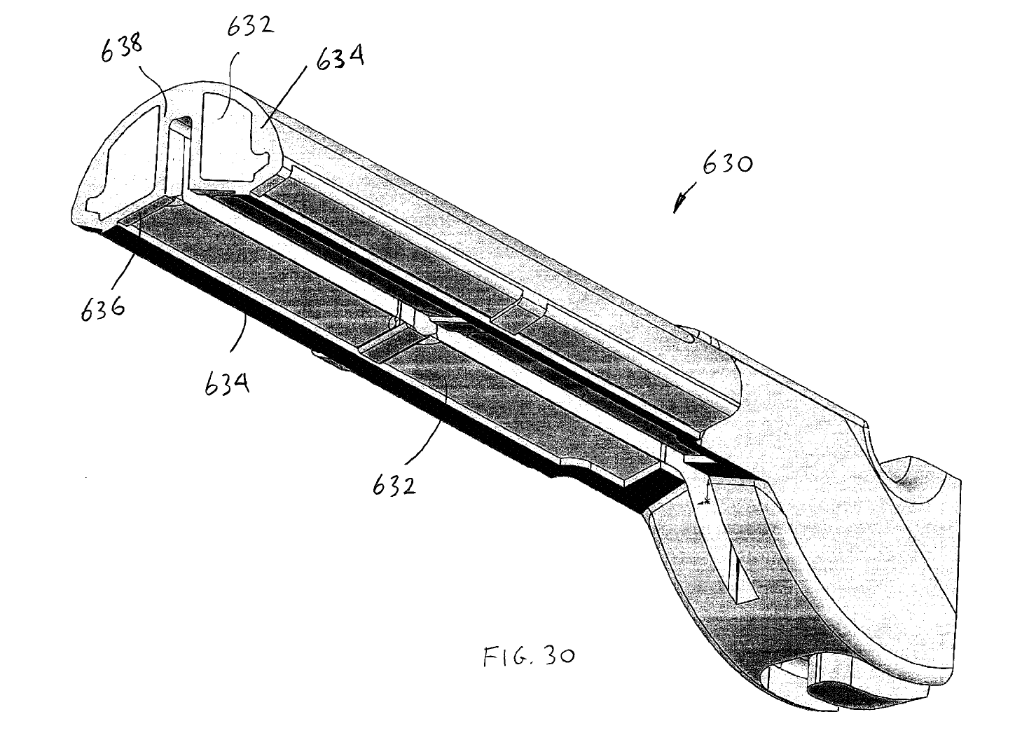

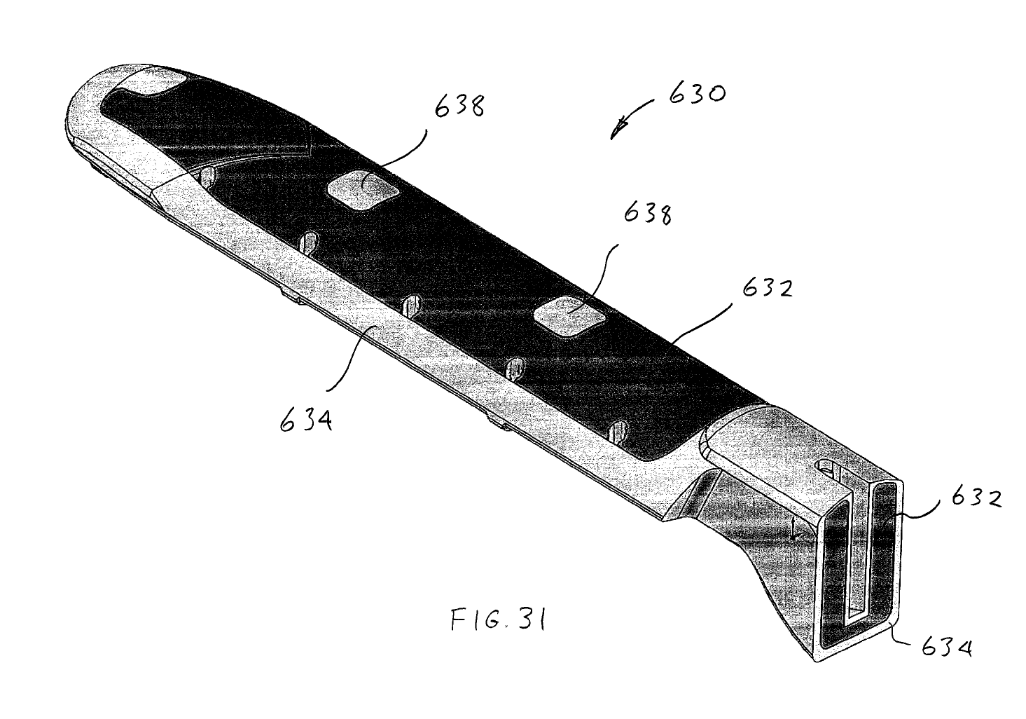

Referring to FIG. 27, jaw structure 630 (of FIG. 25) is shown before the overmolding process (i.e. just central body 632. FIG. 28 shows the same view as FIG. 27 after the overmolding process. Similarly, FIG. 29 shows a different perspective view of central body 632 before the overmolding process, and FIG. 30 shows the same view after. The tip of jaw assembly 630 is shown in cross-section to reveal how cross straps 666 and overmolded plugs 638 cooperate to capture portions of central body 632. FIG. 31 shows an opposite, upper view of overmolded jaw assembly 630. The proximal base of jaw assembly 630 is shown in cross-section to show how insulating overmolded portion 634 surrounds that portion of central body 632.

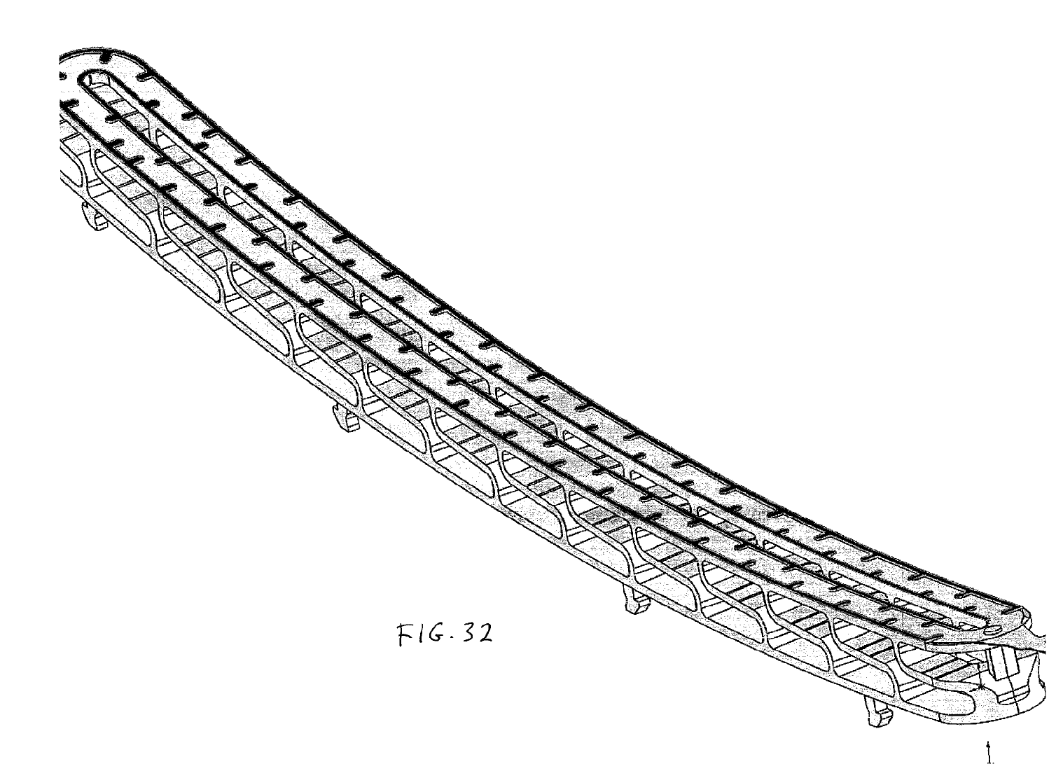

Referring to FIG. 32, another embodiment is shown having peripheral standoff members over the electrode edges with inwardly protruding fingers.

Advantages to the above arrangements include the ability to manage steam egress during application of the tissue seal. Undesirable thermal spread through the target tissue may also be better managed with the above configurations. Better tissue control may also be achieved during clamping and cutting, as the raised lip and/or cross straps may inhibit tissue migration or shifting better than the individual standoff members of the prior art.

Unless defined otherwise, all technical terms used herein have the same meanings as commonly understood by one of ordinary skill in the art of surgery, including electrosurgery. Specific methods, devices, and materials are described in this application, but any methods and materials similar or equivalent to those described herein can be used in the practice of the present invention. While embodiments of the invention have been described in some detail and by way of illustrations, such illustration is for purposes of clarity of understanding only, and is not intended to be limiting. Various terms have been used in the description to convey an understanding of the invention; it will be understood that the meaning of these various terms extends to common linguistic or grammatical variations or forms thereof. It will also be understood that when terminology referring to devices or equipment, that these terms or names are provided as contemporary examples, and the invention is not limited by such literal scope. Terminology that is introduced at a later date that may be reasonably understood as a derivative of a contemporary term or designating of a hierarchal subset embraced by a contemporary term will be understood as having been described by the now contemporary terminology. Further, while some theoretical considerations may have been advanced in furtherance of providing an understanding of the technology, the appended claims to the invention are not bound by such theory. Moreover, any one or more features of any embodiment of the invention can be combined with any one or more other features of any other embodiment of the invention, without departing from the scope of the invention. Still further, it should be understood that the invention is not limited to the embodiments that have been set forth for purposes of exemplification, but is to be defined only by a fair reading of claims appended to the patent application, including the full range of equivalency to which each element thereof is entitled.

1. An electrosurgical device, comprising a jaw assembly structure which includes an upper jaw assembly, and a lower jaw assembly each having at least one electrode wherein at least one standoff member is provided on at least one of the upper jaw assembly and the lower jaw assembly.

2. The electrosurgical device of claim 1, wherein the standoff member is a single, preferable unitary, U-shaped standoff member provided on a jaw of the upper jaw assembly or the lower jaw assembly, so as to maintain a predetermined gap between upper and lower electrodes of the upper and lower jaw assembly, respectively.

3. The electrosurgical device of claim 1, wherein the standoff member comprises a longitudinal slot provided down through its middle so as to allow a cutting element such as a knife to be advanced through a material such as a tissue grasped between the jaws.

4. The electrosurgical device of claim 1, wherein at least one, or both, of the upper jaw assembly and lower jaw assembly comprises a jaw arm, a carrier and electrodes wherein the carrier is provided with a longitudinal, optionally dove-tailed, ridge along its surface, and the jaw arm is provided with a mating, optionally dove-tailed, slot along its lower surface for receiving the ridge.

5. The electrosurgical device of claim 4, wherein at least one, or optionally two slots, which may optionally be dove-tailed, are provided in a surface of the carrier for securing electrodes therein, wherein the electrodes may optionally have a dove-tail shape.

6. The electrosurgical device of claim 4, wherein the standoff member is formed integrally with a center portion of the carrier, preferably the lower carrier.

7. The electrosurgical device of claim 1, wherein pivotable vertebrae are provided for allowing the jaw assembly structure to articulate.

8. The electrosurgical device of claim 1, wherein a U-shaped region of the standoff resides between upper electrodes and lower electrodes so as to keep the upper and lower electrodes a uniform distance apart of about 0.125 to 0.225 mm, or about 0.125 to 0.175 mm, or about 0.150 to 0.175 mm.

9. The electrosurgical device of claim 1, wherein one of the jaw assemblies is provided with a central conductive body which is overmolded with a non-conductive portion including a raised lip.

10. The electrosurgical device of claim 9, wherein the raised lip extends around the periphery of the conductive body and covers an edge portion of the face of the conductive body, the overmolded lip being configured to be interposed between an upper electrode or conductive body and a lower electrode or lower jaw when the upper and lower jaws are in a closed position.

11. The electrosurgical device of claim 9, wherein cross straps are provided across portions of an electrode face.

12. The electrosurgical device of claim 11, wherein overmolded plugs are connected with the cross straps which overmolded plugs pass through the central conductive body.

13. The electrosurgical device of claim 1, comprising peripheral standoff members over electrode edges with inwardly protruding fingers.

14. An electrosurgical device, comprising a jaw assembly structure which includes an upper jaw assembly, and a lower jaw assembly each having at least one electrode wherein at least one standoff member is provided on at least one of the upper jaw assembly and the lower jaw assembly,
wherein one of the jaw assemblies is provided with a central conductive body which is overmolded with a non-conductive portion including a raised lip,
wherein cross straps are provided across portions of an electrode face,
wherein overmolded plugs are connected with the cross straps which overmolded plugs pass through the central conductive body.

15. The electrosurgical device of claim 14, wherein the raised lip extends around the periphery of the conductive body and covers an edge portion of the face of the conductive body, the overmolded lip being configured to be interposed between an upper electrode or conductive body and a lower electrode or lower jaw when the upper and lower jaws are in a closed position.

16. The electrosurgical device of claim 14, wherein the standoff member is a single, preferably unitary, U-shaped standoff member provided on a jaw of the upper jaw assembly or the lower jaw assembly, so as to maintain a predetermined gap between upper and lower electrodes of the upper and lower jaw assembly, respectively.

17. The electrosurgical device of claim 14, wherein the standoff member comprises a longitudinal slot provided down through its middle so as to allow a cutting element such as a knife to be advanced through a material such as a tissue grasped between the jaws.

18. The electrosurgical device of claim 14, wherein at least one, or both, of the upper jaw assembly and lower jaw assembly comprises a jaw arm, a carrier and electrodes wherein the carrier is provided with a longitudinal, optionally dove-tailed, ridge along its surface, and the jaw arm is provided with a mating, optionally dove-tailed, slot along its lower surface for receiving the ridge.

19. The electrosurgical device of claim 18, wherein at least one, or optionally two slots, which may optionally be dove-tailed, are provided in a surface of the carrier for securing electrodes therein, wherein the electrodes may optionally have a dove-tail shape.

20. The electrosurgical device of claim 18, wherein the standoff member is formed integrally with a center portion of the carrier, preferably the lower carrier.
